(19) United States
(12) Patent Application Publication
(10) Pub. No.: US 2013/0244185 A9
(43) Pub. Date: Sep. 19, 2013

(54) RADIATION-SENSITIVE RESIN COMPOSITION, METHOD FOR FORMING RESIST PATTERN, ORGANIC ACID AND ACID GENERATING AGENT

(71) Applicants: Yasuhiko MATSUDA, Tokyo (JP); Takanori Kawakami, Tokyo (JP)

(72) Inventors: Yasuhiko MATSUDA, Tokyo (JP); Takanori Kawakami, Tokyo (JP)

(73) Assignee: JSR Corporation, Tokyo (JP)

(21) Appl. No.: 13/615,842

(22) Filed: Sep. 14, 2012

Related U.S. Application Data
(63) Continuation of application No. PCT/JP2011/056111, filed on Mar. 15, 2011.

(30) Foreign Application Priority Data
Mar. 17, 2010 (JP) 2010-061720

(51) Int. Cl. C07C 321/10; G03F 7/20

(52) U.S. Cl. 430/325; 560/117; 560/126; 562/100; 562/113

(57) ABSTRACT

A radiation-sensitive resin composition includes an acid generating agent to generate an organic acid by irradiation with a radioactive ray. The organic acid has a cyclic hydrocarbon group and an organic group including a bond that is cleavable by an acid or a base to produce a polar group. The organic acid is preferably represented by a following formula (I). Z represents an organic acid group. R¹ represents an alkanediyl group, wherein a part or all of hydrogen atoms of the alkanediyl group represented by R¹ are optionally substituted by a fluorine atom. X represents a single bond, O, OCO, COO, CO, SO₃ or SO₂. R² represents a cyclic hydrocarbon group. R³ represents a monovalent organic group having a functional group represented by a following formula (x). n is an integer of 1 to 3.

Z—R¹—X—R²—(R³)ₙ (I)

—R³¹-G-R¹³ (x)

### CROSS-REFERENCE TO RELATED APPLICATIONS

The present application is a continuation application of International Application No. PCT/JP2011/056111, filed Mar. 15, 2011, which claims priority to Japanese Patent Application No. 2010-061720, filed Mar. 17, 2010. The contents of these applications are incorporated herein by reference in their entirety.

### BACKGROUND OF THE INVENTION

1. Field of the Invention

The present invention relates to a radiation-sensitive resin composition, a method for forming a resist pattern, an organic acid and an acid generating agent.

2. Discussion of the Background

In the field of microfabrication represented by manufacturing of integrated circuit elements, in order to obtain higher integrity, microfabrication at a level of no greater than 0.10 μm has been recently required. As radioactive rays used for such microfabrication, for example, a KrF excimer laser (wavelength: 248 nm), an ArF excimer laser (wavelength: 193 nm), an F2 excimer laser (wavelength: 157 nm), EUV light (wavelength: 13 nm), an electron beam and the like have attracted attention.

With attention to such radioactive rays, a number of photoresist materials have been proposed. Examples of the photoresist materials include compositions which contain a component having an acid-dissociable group and a component that generates an acid (acid generating agent) by irradiation with a radioactive ray (exposure), and utilize a chemical amplification effect between the components, and the like. It has been reported that use of an acid generating agent having an anion made bulky by introduction of an adamantane structure, a norbornene structure, and the like in a positive type photoresist material using an ArF excimer laser, for example, enables an appropriate control of a diffusion length of an acid generated and favorable development properties such as MEEF (Mask Error Enhancement Factor), LWR (Line Width Roughness) and ER (Edge Roughness) (see PCT International Publication No. 2009/051088 and Japanese Unexamined Patent Application, Publication No. 2004-002252).

Under such circumstances, in the field of microfabrication, it has been required to form a very fine resist pattern having a further smaller line width of about 45 nm and the like. Examples of strategies for enabling formation of the very fine resist pattern include shortening a light source wavelength in a lithography device, an increase of numerical aperture (NA) of a lens, and the like. However, shortening a light source wavelength requires a new lithography device which causes an increase in cost. In addition, an increase of numerical aperture of a lens leads to a decrease of a depth of focus although enhancement in resolution can be realized.

As a lithography technique for solving the problems described above, a liquid immersion lithography process has been known.

According to the liquid immersion lithography process, even if a conventional exposure light is used, a similar effect to the case in which a wavelength of exposure light is shortened can be obtained, which is advantageous in cost, and a resist pattern excellent in resolving ability and depth of focus can be formed. As various compositions capable of being adopted to the liquid immersion lithography, for example, PCT International Publication No. 2005/069076 discloses a conventional resist film and an upper layer film for liquid immersion lithography. In addition, PCT International Publication No. 2007/116664 discloses a composition containing a fluorine-containing polymer and a resin having an acid-labile group as a composition for a resist film which requires no upper layer film for liquid immersion lithography.

### SUMMARY OF THE INVENTION

According to one aspect of the present invention, a radiation-sensitive resin composition includes an acid generating agent to generate an organic acid by irradiation with a radioactive ray. The organic acid has a cyclic hydrocarbon group and an organic group including a bond that is cleavable by an acid or a base to produce a polar group.

According to another aspect of the present invention, a method for forming a resist pattern includes providing the radiation-sensitive resin composition on a substrate to form a photoresist film. The formed photoresist film is exposed through an immersion liquid. The exposed photoresist film is developed to form a resist pattern.

According to further aspect of the present invention, an organic acid is represented by a following formula (I) or a salt thereof.

Z—R¹—X—R²—(R³)ₙ (I)

Z represents an organic acid group. R¹ represents an alkanediyl group, wherein a part or all of hydrogen atoms of the alkanediyl group represented by R¹ are optionally substituted by a fluorine atom. X represents a single bond, O, OCO, COO, CO, SO₃ or SO₂. R² represents a cyclic hydrocarbon group. R³ represents a monovalent organic group having a functional group represented by a following formula (x). n is an integer of 1 to 3, wherein in a case where R³ is present in a plurality of number, R³s present in the plurality of number are a same or different.

—R³¹-G-R¹³ (x)

R³¹ represents a single bond or a bivalent linking group. G represents an oxygen atom, an imino group, —NR¹³¹—, —CO—O—*, —O—CO—* or —SO₂—O—*, wherein the oxygen atom represented by G excludes an oxygen atom directly bonded to a carbonyl group or a sulfone group. Each of R¹³¹ and R¹³ represents an acid-dissociable group or a base-dissociable group. “*” denotes a site bound to R¹³.

According to further aspect of the present invention, an acid generating agent generates an organic acid represented by a following formula (I) by irradiation with a radioactive ray.

Z—R¹—X—R²—(R³)ₙ (I)

Z represents an organic acid group. R¹ represents an alkanediyl group, wherein a part or all of hydrogen atoms of the alkanediyl group represented by R¹ are optionally substituted by a fluorine atom. X represents a single bond, O, OCO, COO, CO, SO₃ or SO₂. R² represents a cyclic hydrocarbon group. R³ represents an organic group having a monovalent functional group represented by a following formula (x). n is an integer of 1 to 3, wherein in a case where R³ is present in a plurality of number, R³s present in the plurality of number are a same or different.

—R³¹-G-R¹³ (x)

In the formula (x), R³¹ represents a single bond or a bivalent linking group. G represents an oxygen atom, an imino group, —NR¹³¹—, —CO—O—*, —O—CO—* or —SO₂—O—*, wherein the oxygen atom represented by G excludes an oxygen atom directly bonded to a carbonyl group or a sulfone group. Each of R¹³¹ and R¹³ represents an acid-dissociable group or a base-dissociable group. “*” denotes a site bound to R¹³.

### DESCRIPTION OF THE EMBODIMENTS

One aspect of embodiments of the present invention provides a radiation-sensitive resin composition containing,

(A) an acid generating agent that generates an organic acid by irradiation with a radioactive ray (hereinafter, may be also referred to as acid generating agent (A)), wherein

the organic acid has a cyclic hydrocarbon group and an organic group including a bond that is cleavable by an acid or a base to produce a polar group.

In the composition, an organic acid generated from the acid generating agent (A) upon irradiation with a radioactive ray has a cyclic hydrocarbon group and an organic group including a bond that is cleavable by an acid or a base to produce a polar group (hereinafter, may be also referred to as “cleavable bond”), so that cleavage of the cleavable bond in the organic group occurs by the organic acid generated per se or an alkaline developer and an affinity to the alkaline developer is enhanced. As a result, aggregation of the organic acid during a development step is inhibited, thereby preventing generation of development defects. In addition, the organic acid has a bulky cyclic hydrocarbon group having a high content of carbon, so that an appropriately short diffusion length of an organic acid in a photoresist film is attained, thereby leading to improvement of MEEF and LWR in a pattern.

The organic acid is preferably represented by the following formula (I):

Z—R¹—X—R²—(R³)ₙ (I)

in the formula (I), Z represents an organic acid group; R¹ represents an alkanediyl group, wherein a part or all of hydrogen atom in the alkanediyl group are optionally substituted by a fluorine atom; X represents a single bond, O, OCO, COO, CO, SO₃ or SO₂; R² represents a cyclic hydrocarbon group; R³ represents a monovalent organic group having a functional group represented by the following formula (x); n is an integer of 1 to 3, and wherein provided that R³ is present in a plurality of number, R³ present in a plurality of number may be the same or different,

—R³¹-G-R¹³ (x)

in the formula (x), R³¹ represents a single bond or a bivalent linking group; G represents an oxygen atom, an imino group, —NR¹³¹—, —CO—O—*, —O—CO—* or —SO₂—O—*, wherein the oxygen atom excludes one directly linked to a carbonyl group or a sulfone group; R¹³¹ and R¹³ represent an acid-dissociable group or a base-dissociable group; and “*” denotes a site bound to R¹³.

Due to having the specific structure, the organic acid enables an affinity to an alkaline developer in a development step and an appropriately short diffusion length of the organic acid in a photoresist film to be highly balanced, whereby development defects are prevented, and superior MEEF and LWR can be attained.

The Z is preferably SO₃H. Employing a sulfonic acid group as the organic acid group in the organic acid enables a sufficiently high strength of the acid, whereby sufficient sensitivity as a photoresist can be achieved.

The R¹ is preferably represented by the following formula (1) :

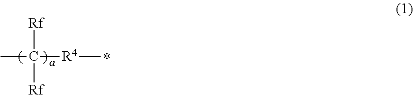

in the formula (1), Rfs each independently represent a hydrogen atom, a fluorine atom, or an alkyl group in which a part or all of hydrogen atoms are substituted by a fluorine atom; R⁴ represents an alkanediyl group; a is an integer of 1 to 8, wherein provided that a is 2 or more, Rfs present in a plurality of number may be the same or different, but any case where all the Rfs are a hydrogen atom is excluded; and “*” denotes a site bound to X.

Introduction of a fluorine atom having a high electron-withdrawing property into an alkanediyl group adjacent to the organic acid group enables the strength of the organic acid to be enhanced and the sensitivity as a photoresist to be further improved.

The R³ optionally includes a structure represented by the following formula (2):

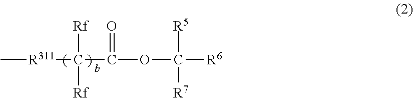

in the formula (2), R³¹¹ represents a single bond or a bivalent linking group; Rf is as defined in connection with the above formula (1); R⁵ to R⁷ each independently represent an alkyl group having 1 to 4 carbon atoms or an alicyclic hydrocarbon group having 4 to 20 carbon atoms; R⁶ and R⁷ may taken together represent a bivalent alicyclic hydrocarbon group having 4 to 20 carbon atoms together with the carbon atom to which R⁶ and R⁷ bond; b is an integer of 0 to 8, wherein provided that b is 2 or more, the Rfs present in a plurality of number may be the same or different, but any case where all the Rfs are a hydrogen atom is excluded.

The R³ includes a structure represented by the above formula (2), so that a bulky anion moiety can be acid-dissociable, and R³ dissociates by an organic acid generated, thereby resulting in enhancement of an affinity of the organic acid to an alkaline developer.

The R³ optionally includes a structure represented by the following formula (3) or (4):

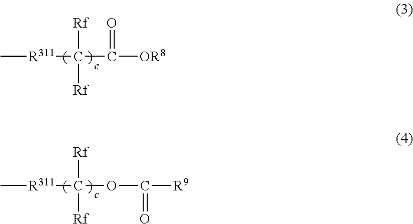

in the formulae (3) and (4), R³¹¹ is as defined in connection with the above formula (2) ; Rf is as defined in connection with the above formula (1) ; R⁸ represents an alkyl group having 1 to 10 carbon atoms in which a part or all of hydrogen atoms are substituted by a fluorine atom or a group represented by the following formula (5), (6) or (7); R⁹ represents an alkyl group having 1 to 10 carbon atoms in which a part or all of hydrogen atoms are substituted by a fluorine atom; c is an integer of 0 to 4, wherein provided that c is 2 or more, the Rfs present in a plurality of number may be the same or different, but any case where all the Rfs are a hydrogen atom is excluded:

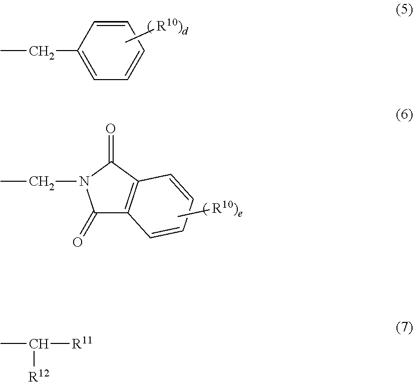

in the formulae (5) and (6), R¹⁰ each independently represents a halogen atom, an alkyl group, an alkoxy group, an acyl group or an acyloxy group having 1 to 10 carbon atoms; d is an integer of 0 to 5; e is an integer of 0 to 4, wherein provided that R¹⁰ is present in a plurality of number, the R¹⁰s present in a plurality of number may be the same or different;
in the formula (7), R¹¹ and R¹² each independently represent a hydrogen atom or an alkyl group having 1 to 10 carbon atoms, and wherein R¹¹ and R¹² may taken together represent an alicyclic structure having 4 to 20 carbon atoms together with the carbon atom to which R¹¹ and R¹² bond.

The R³ includes a structure represented by the above formula (3) or (4), so that a bulky anion moiety can be base-dissociable, and thus R³ dissociates by an alkaline developer, whereby an affinity of the organic acid to the alkaline developer can be enhanced.

The R² is preferably represented by the following formula (8), (9) or (10) :

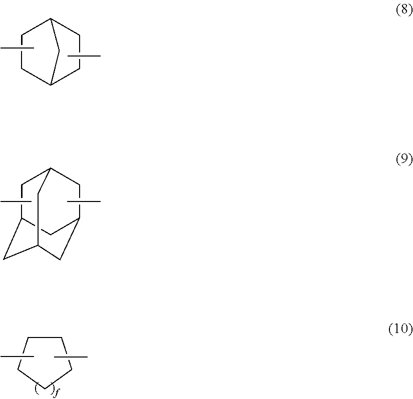

in the formula (10), f is an integer of 1 to 10.

Introduction of such a bulky structure as one represented by the above formulae (8) to (10) into the organic acid enables a content of carbon in the organic acid to increase and a more appropriate diffusion length of the organic acid to be attained in a photoresist film.

The R² described above preferably represents a polycyclic hydrocarbon group. When the R² described above represents a polycyclic hydrocarbon group, the content of carbon in the organic acid is increased, whereby a diffusion length of the organic acid in a photoresist film can be suitably adjusted.

The acid generating agent (A) is preferably a sulfonium salt compound or an iodonium salt compound of the organic acid represented by the above formula (I). The acid generating agent (A) having the salt compound form described above enables deprotection reaction in a sulfo group by a radioactive ray to be facilitated, thereby enhancing a radiation-sensitive property of the acid generating agent (A).

Another aspect of the embodiments of the present invention provides a method for forming a resist pattern, the method including:

(1) a step of forming a photoresist film on a substrate using the composition,

(2) a step of exposing the formed photoresist film through an immersion liquid, and

(3) a step of developing the exposed photoresist film to form a resist pattern.

In the method for formation, the composition is used as a photoresist composition, so that a resist pattern favorable in MEEF and LWR can be formed while development defects in the development step can be inhibited.

Still another aspect of the embodiments of the present invention provides an organic acid represented by the following formula (I) or a salt thereof:

Z—R¹—X—R²—(R³)ₙ (I)

in the formula (I), Z represents an organic acid group; R¹ represents an alkanediyl group, wherein a part or all of hydrogen atoms of the alkanediyl group are optionally substituted by a fluorine atom; X represents a single bond, O, OCO, COO, CO, SO₃ or SO₂; R² represents a cyclic hydrocarbon group; R³ represents a monovalent organic group having a functional group represented by the following formula (x); n is an integer of 1 to 3, and wherein provided that R³ is present in a plurality of number, the R³s present in a plurality of number may be the same or different:

—R³¹-G-R¹³ (x)

in the formula (x), R³¹ represents a single bond or a bivalent linking group; G represents an oxygen atom, an imino group, —NR¹³¹—, —CO—O—*, —O—CO—* or —SO₂—O—*, wherein the oxygen atom excludes one directly linked to a carbonyl group or a sulfone group; R¹³¹ and R¹³ represent an acid-dissociable group or a base-dissociable group; and “*” denotes a site bound to R¹³.

Due to having a structure represented by the above formula (I), the organic acid or the salt thereof can be suitably used as the acid generating agent or a precursor thereof in the composition.

Yet another aspect of the embodiments of the present invention provides an acid generating agent that generates an organic acid represented by the following formula (I) by irradiation with a radioactive ray:

Z—R¹—X—R²—(R³)ₙ (I)

in the formula (I), Z represents an organic acid group; R¹ represents an alkanediyl group, wherein a part or all of hydrogen atoms of the alkanediyl group are optionally substituted by a fluorine atom; X represents a single bond, O, OCO, COO, CO, SO₃ or SO₂; R² represents a cyclic hydrocarbon group; R³ represents a monovalent organic group having a functional group represented by the following formula (x); n is an integer of 1 to 3, and wherein provided that the R³ is present in a plurality of number, the R³s present in a plurality of number may be the same or different:

—R³¹-G-R¹³ (x)

in the formula (x), R³¹ represents a single bond or a bivalent linking group; G represents an oxygen atom, an imino group, —NR¹³¹—, —CO—O—*, —O—CO—* or —SO₂—O—*, wherein the oxygen atom excludes one directly bonded to a carbonyl group or a sulfone group; R¹³¹ and R¹³ represent an acid-dissociable group or a base-dissociable group; and “*” denotes a site bound to R¹³.

The acid generating agent generates an organic acid represented by the above formula (I), so that the acid generating agent can be suitably used in a radiation-sensitive resin composition.

According to the embodiment of the present invention, a radiation-sensitive resin composition excellent in MEEF and LWR also in a liquid immersion lithography process and capable of forming a resist pattern having reduced generation of development defects such as a blob in which development residues deposit on the patterns formed can be provided. The embodiments will now be described in detail.

### <Radiation-Sensitive Resin Composition>

The radiation-sensitive resin composition of the embodiment of the present invention contains (A) an acid generating agent. In addition, the radiation-sensitive resin composition of the embodiment of the present invention may contain (B) a polymer and (C) a fluorine atom-containing polymer described later as suitable components. Furthermore, the radiation-sensitive resin composition of the embodiment of the present invention may contain other optional components. Hereinafter, each component will be explained in detail.

### <(A) Acid Generating Agent>

The acid generating agent (A) generates an organic acid having a cyclic hydrocarbon group and an organic group including a cleavable bond by irradiation with a radioactive ray. The acid generating agent (A) typically has a moiety corresponding to an organic acid ion and a counter ion moiety corresponding thereto. Such an acid generating agent generates an organic acid by exposure, so that photoresist sensitivity of the composition is enhanced upon exposure, thereby enabling development defects in the development step to be prevented.

The organic acid is not particularly limited as long as the organic acid has a cyclic hydrocarbon group and an organic group including a cleavable bond. Each position of the cyclic hydrocarbon group, the organic group including a cleavable bond, and the organic acid group in the entire structure of the organic acid is not particularly limited. The organic acid generated from the acid generating agent (A) has a cyclic hydrocarbon group and thus has a high content of carbon of the organic acid, as a result, an appropriate diffusion length in a resin can be provided. In addition, the organic acid has a cleavable bond, so that the cleavable bond is cleaved by a developer solution in the development step, a polar group is generated and thus the organic acid that exhibited comparatively high hydrophobicity resulting from the cyclic hydrocarbon group consequently exhibits an affinity to the developer solution. As a result, aggregation in the development step is inhibited, thereby enabling inhibition of development defects.

The organic acid group included in the organic acid is not particularly limited as long as it is a group showing acidity, and examples thereof include SO₃H (sulfonic acid group), COOH (carboxyl group), and the like. Examples of the cyclic hydrocarbon group included in the organic acid include a monocyclic hydrocarbon group, a polycyclic hydrocarbon group, a combination thereof, and the like. Introduction of the cyclic hydrocarbon group enables bulkiness to be imparted to the organic acid ion moiety to permit appropriate adjustment of a diffusion length. In addition, although the position of the cyclic hydrocarbon group which the organic acid has is not particularly limited as described above, in light of ease in cleavage of the cleavable bond, the cyclic hydrocarbon group is preferably positioned as a linking group between the organic group including the cleavable bond and the organic acid group. The organic group including the cleavable bond is exemplified by a group represented by the above formula (x).

In the above formula (x), R³¹ represents a single bond or a bivalent linking group; G represents an oxygen atom, an imino group, —NR¹³¹—, —CO—O—*, —O—CO—* or —SO₂—O—*, wherein the oxygen atom excludes one directly linked to a carbonyl group and a sulfone group; R¹³¹ and R¹³ represent an acid-dissociable group or a base-dissociable group; and “*” denotes a site bound to R¹³. In other words, in the above formula (x), -G-R¹³ is a group derived by modifying a hydroxyl group, an amino group, a carboxyl group or a sulfo group with an acid-dissociable group or a base-dissociable group.

It is to be noted that the “acid-dissociable group” refers to a group that substitutes for a hydrogen atom in a polar functional group and is dissociated in the presence of an acid, and the “base-dissociable group” refers to a group that substitutes for a hydrogen atom in a polar functional group and is dissociated in the presence of a base.

Examples of the bivalent linking group represented by the R³¹ include an ether group, an ester group, a carbonyl group, a bivalent chain hydrocarbon group having 1 to 30 carbon atoms, a bivalent alicyclic hydrocarbon group having 3 to 30 carbon atoms, a bivalent aromatic hydrocarbon group having 6 to 30 carbon atoms or a bivalent group in combination thereof, and the like.

Examples of the bivalent chain hydrocarbon group having 1 to 30 carbon atoms represented by the R³¹ include linear alkanediyl groups such as a methylene group, an ethylene group, a 1,2-propylene group, a 1,3-propylene group, a tetramethylene group, a pentamethylene group, a hexamethylene group, a heptamethylene group, an octamethylene group, a nonamethylene group, a decamethylene group, an undecamethylene group, a dodecamethylene group, a tridecamethylene group, a tetradecamethylene group, a pentadecamethylene group, a hexadecamethylene group, a heptadecamethylene group, an octadecamethylene group, a nonadecamethylene group, an icosalene group; branched alkanediyl groups such as a 1-methyl-1,3-propylene group, a 2-methyl-1,3-propylene group, a 2-methyl-1,2-propylene group, a 1-methyl-1,4-butylene group, a 2-methyl-1,4-butylene group, an ethylidene group, a 1-propylidene group, a 2-propylidene group, and the like.

Examples of the bivalent alicyclic hydrocarbon group having 3 to 30 carbon atoms represented by the R³¹ include monocyclic cycloalkanediyl groups having 3 to 10 carbon atoms such as a 1,3-cyclobutylene group, a 1,3-cyclopentylene group, a 1,4-cyclohexylene group, a 1,5-cyclooctylen group; polycyclic cycloalkanediyl groups such as a 1,4-norbornylene group, a 2,5-norbornylene group, a 1,5-adamantylene group, a 2,6-adamantylene group, and the like.

Examples of the bivalent aromatic hydrocarbon group having 6 to 30 carbon atoms represented by the R³¹ include arylene groups such as a phenylene group, a tolylene group, a naphthylene group, a phenanthtylene group, an anthrylene group, and the like.

The bivalent linking group represented by the R³¹ may have a substituent. Examples of the substituent include a halogen atom, —R^{S1}, —R^{S2}—O—R^{S1}, —R^{S2}—CO—R^{S1}, —R^{S2}—CO—OR^{S1}, —R^{S2}—O—CO—R^{S1}, —R^{S2}—OH, —R^{S2}—CN, and the like. R^{S1} represents an alkyl group having 1 to 30 carbon atoms, a cycloalkyl group having 3 to 20 carbon atoms or an aryl group having 6 to 30 carbon atoms. It is to be noted that a part or all of hydrogen atoms these groups have may be substituted by a fluorine atom. R^{S2} represents a single bond, an alkanediyl group having 1 to 10 carbon atoms, a cycloalkanediyl group having 3 to 20 carbon atoms, or an arylene group having 6 to 30 carbon atoms. It is to be noted that a part or all of hydrogen atoms these groups have may be substituted by a fluorine atom.

Examples of the alkyl group having 1 to 30 carbon atoms represented by the R^{S1} include a methyl group, an ethyl group, a 1-propyl group, a 2-propyl group, a 1-butyl group, a 2-butyl group, a 2-(2-methylpropyl) group, a 1-pentyl group, a 2-pentyl group, a 3-pentyl group, a 1-(2-methylbutyl) group, a 1-(3-methylbutyl) group, a 2-(2-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(2-methylpentyl) group, a 2-(3-methylpentyl) group, a 2-(4-methylpentyl) group, a 3-(2-methylpentyl) group, a 3-(3-methylpentyl) group, and the like.

Examples of the cycloalkyl group having 3 to 20 carbon atoms represented by the R^{S1} include a cyclopentyl methyl group, a 1-(1-cyclopentylethyl) group, a 1-(2-cyclopentylethyl) group, a cyclohexylmethyl group, a 1-(1-cyclohexylethyl) group, a 1-(2-cyclohexylethyl group), a cycloheptyl methyl group, a 1-(1-cycloheptyl ethyl) group, a 1-(2-cycloheptyl ethyl) group, a 2-norbornyl group, and the like.

Examples of the aryl group having 6 to 30 carbon atoms represented by the R^{S1} include a phenyl group, a benzyl group, a phenylethyl group, a phenylpropyl group, a phenylcyclohexyl group, and the like.

Examples of the alkanediyl group having 1 to 30 carbon atoms, the cycloalkanediyl group having 3 to 20 carbon atoms and the arylene group having 6 to 30 carbon atoms represented by the R^{S2} include groups derived by removing one hydrogen atom from the above-exemplified groups, and the like.

The organic acid is preferably represented by the above formula (I). Due to having the specific structure, the organic acid enables an affinity to an alkaline developer in a development step and an appropriately short diffusion length of the organic acid in a photoresist film to be highly balanced, whereby development defects are further prevented, and superior MEEF and LWR can be attained.

In the above formula (I), Z represents an organic acid group; R¹ represents an alkanediyl group, wherein a part or all of hydrogen atoms of the alkanediyl group may be substituted by a fluorine atom; X represents a single bond, O, OCO, COO, CO, SO₃ or SO₂; R² represents a cyclic hydrocarbon group; R³ represents a monovalent organic group having a functional group represented by the following formula (x); n is an integer of 1 to 3, and wherein provided that R³ is present in a plurality of number, the R³s present in a plurality of number may be the same or different.

Examples of the organic acid group represented by the Z include the organic acid groups described above, and the like. Of these, SO₃H (sulfonic acid group) is preferable in terms of enhancement in resist sensitivity.

The alkanediyl group represented by the R¹ is preferably an alkanediyl group having 1 to 12 carbon atoms, more preferably an alkanediyl group having 1 to 6 carbon atoms and particularly preferably an alkanediyl group having 1 to 4 carbon atoms. In addition, the alkanediyl group may have a linking group such as an oxygen atom or a sulfur atom. In the alkanediyl group represented by the R¹, a part or all of hydrogen atoms may be substituted by a fluorine atom, and an alkanediyl group in which 30% to 100% of hydrogen atoms are substituted by a fluorine atom is particularly preferable. A carbon atom substituted with a fluorine atom is more preferably a carbon atom bound to Z.

R¹ is preferably a group represented by the above formula (1). Introduction of a fluorine atom having a high electron-withdrawing property into an alkanediyl group adjacent to the organic acid group enables the strength of the organic acid to be further enhanced and the sensitivity as a photoresist to be further improved.

In the above formula (1), Rfs each independently represent a hydrogen atom, a fluorine atom, or an alkyl group in which a part or all of hydrogen atoms are substituted by a fluorine atom; R⁴ represents an alkanediyl group; a is an integer of 1 to 8, wherein provided that a is or more, the Rfs present in a plurality of number may be the same or different, but any case where all the Rfs are a hydrogen atom is excluded; and “*” denotes a site bound to X.

The Rf is preferably a fluorine atom or a trifluoromethyl group; a is preferably an integer of 1 to 3; and R⁴ is preferably an alkanediyl group having 1 to 3 carbon atoms.

R¹ is preferably a group represented by the following formula

*—(CF₂)ₙ—

*—CF₂CF₂ (CH₂)ₙ—

*—CF₂CHF(CH₂)ₙ— or

*—CF(CF₃)(CH₂)ₙ—

In the above formula, n is each independently an integer of 1 to 4; and “*” denotes a site bound to Z.

X is preferably OCO or COO in light of easiness of synthesis, chemical stability, and the like.

R² represents a cyclic hydrocarbon group having 3 to 30 carbon atoms. Examples of the cyclic hydrocarbon group having 3 to 30 carbon atoms include a cyclopropylene group, a cyclobutylene group, a cyclopentylene group, a cyclohexylene group, a bornylene group, a norbornylene group, an adamantylene group, a pinanylene group, a tsuyoylene group, a calylene group, a camphornylene group, a methylcyclopropylene group, a methylcyclobutylene group, a methylcyclopentylene group, a methylcyclohrxylene group, a methylbornylene group, a methylnorbornylene group and a methyladamantylene group, and the like.

The cyclic hydrocarbon group may be substituted, and examples of a substituent include a halogen atom, a hydroxyl group, a thiol group, an aryl group, an alkenyl group, organic groups that include a hetero atom (a halogen atom, an oxygen atom, a nitrogen atom, a sulfur atom, a phosphorus atom, a silicon atom, etc.), and the like. Further, a keto group derived by substituting two hydrogen atoms on the same carbon in the aforementioned hydrocarbon group by one oxygen atom can be exemplified. The number of the substituent is not limited within a structurally acceptable range.

Examples of the R² include a fluorocyclohexylene group, a hydroxycyclohexylene group, a methoxycyclohexylene group, a methoxycarbonyl cyclohexylene group, a hydroxyadamantylene group, a methoxycarbonyladamantylene group, a hydroxycarbonyladamantylene group, a hydroxymethyl an adamantanemethylene group, and the like.

Of these, R² is preferably a group represented by the above formula (8), (9) or (10). In the above formula (10), f is an integer of 1 to 10. Introduction of such a bulky structure as one represented by the above formulae (8) to (10) into the organic acid enables a content of carbon in the organic acid to increase and a more appropriate diffusion length of the organic acid to be attained in a photoresist film.

Of the cleavable bonds, a bond which is cleavable by an acid to generate a polar group is also hereinafter referred to as “acid cleavable bond”, and a bond which is cleavable by a base to form a polar group is also hereinafter referred to as “base-cleavable bond”.

In the above formula (I), R³ represents a monovalent organic group having an acid-cleavable bond among the cleavable bonds. The organic group is suitably configured as a form in which an acid-dissociable group and a bivalent linking group are bound via the cleavable bond.

With respect to the acid cleavable bond, the R³ is preferably a group including the structure represented by the above formula (2). R³ having such a structure facilitates cleavage of a cleavable bond by an acid.

In the above formula (2), R³¹¹ represent a single bond or a bivalent linking group; Rf is as defined in connection with the above formula (1); R⁵ to R⁷ each independently represent an alkyl group having 1 to 4 carbon atoms or an alicyclic hydrocarbon group having 4 to 20 carbon atoms; in addition, R⁶ and R⁷ may taken together represent a bivalent alicyclic hydrocarbon group having 4 to 20 carbon atoms together with the carbon atom to which R⁶ and R⁷ bond; b is an integer of 0 to 8, and wherein provided that b is 2 or more, Rfs present in a plurality of number may be the same or different, but any case where all the Rfs are a hydrogen atom is excluded.

Examples of the alkyl group having 1 to 4 carbon atoms represented by the R⁵ to R⁷ include a methyl group, an ethyl group, an n-propyl group, an i-propyl group, an n-butyl group, a 2-methylpropyl group, a 1-methylpropyl group, a t-butyl group, and the like. Examples of the alicyclic hydrocarbon group having 4 to 20 carbon atoms represented by the R⁵ to R⁷, and the bivalent alicyclic hydrocarbon group having 4 to 20 carbon atoms which may be formed from R⁶ and R⁷ together with the carbon atom to which R⁶ and R⁷ bond include bridged skeletons such as an adamantane skeleton, a norbornane skeleton, a tricyclodecane skeleton, a tetracyclododecane skeleton; groups having a cycloalkane skeleton such as cyclobutane, cyclopentane, cyclohexane, cycloheptane and cyclooctane; groups derived by substituting these groups with one type or more linear, branched or cyclic alkyl group having 1 to 10 carbon atoms such as a methyl group, an ethyl group, an n-propyl group, an i-propyl group or an n-butyl group, and the like.

The acid cleavable bond binds to R² via a linking group R³¹¹. The bivalent linking group represented by R³¹¹ may be similarly defined to the bivalent linking group represented by the R³¹. R³¹¹ is, provided that b is 0, preferably *—COOR^{31a}—, or *—OCOR^{31a}—. R^{31a} may be similarly defined to the bivalent chain hydrocarbon group represented by R³¹. “*” denotes a site bound to R². Provided that b is present in a plurality of number, the b is preferably an oxygen atom, COO or OCO.

In the above formula (I), R³ may be a monovalent organic group having a base-cleavable bond. The organic group is suitably configured as a form in which a base-dissociable group and a bivalent linking group are bound via a cleavable bond. The base-dissociable group is not particularly limited as long as the properties described above are provided, and examples thereof include, provided that G represents an oxygen atom or an imino group in the above formula (x), a structure represented by the following formula (11), and the structure represented by the above formula (3) or (4).

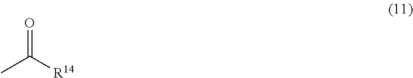

In the above formula (11), R¹⁴ represents a hydrocarbon group having 1 to 10 carbon atoms in which at least one hydrogen atom is substituted by a fluorine atom.

R¹⁴ is preferably, for example, a linear or branched alkyl group having 1 to 10 carbon atoms, or a group in which a part or all of hydrogen atoms of an alicyclic hydrocarbon group having 3 to 20 carbon atoms are substituted by a fluorine atom.

The alkyl group having 1 to 10 carbon atoms represented by the R¹⁴ may be similarly defined to the alkyl group represented by the R^{S1}. The alicyclic hydrocarbon group having 3 to 20 carbon atoms represented by the R¹⁴ may be similarly defined to the cycloalkyl group represented by the R^{S1}.

R¹⁴ is preferably a linear or branched perfluoroalkyl group having 1 to 10 carbon atoms and more preferably a trifluoromethyl group.

The R³ preferably includes a structure represented by the above formula (3) or (4). When the R³ includes a structure represented by the above formula (3) or (4), a bulky anion moiety can be base-dissociable, and an affinity of the organic acid to the alkaline developer upon dissociation of R³ by an alkaline developer can be enhanced.

In the above formulae (3) and (4), R³¹¹ is as defined in connection with the above formula (2); Rf is as defined in connection with the above formula (1); R⁸ represents an alkyl group having 1 to 10 carbon atoms in which a part or all of hydrogen atoms are substituted by a fluorine atom, or a group represented by the above formula (5), (6) or (7); R⁹ represents an alkyl group having 1 to 10 carbon atoms in which a part or all of hydrogen atoms are substituted by a fluorine atom; c is an integer of 0 to 4, wherein provided that c is present in a plurality of number, the Rfs present in a plurality of number may be the same or different, but any case where all the Rfs are a hydrogen atom is excluded. In the above formulae (5) and (6), R¹⁰ each independently represents a halogen atom, an alkyl group having 1 to 10 carbon atoms, an alkoxy group, an acyl group or an acyloxy group; d is an integer of 0 to 5; and e is an integer of 0 to 4, wherein provided that R¹⁰ is present in a plurality of number, the R¹⁰s present in a plurality of number may be the same or different. In the above formula (7), R¹¹ and R¹² each independently represent a hydrogen atom or an alkyl group having 1 to 10 carbon atoms, wherein R¹¹ and R¹² may taken together represent an alicyclic structure having 4 to 20 carbon atoms together with the carbon atom to which R¹¹ and R¹² bond.

The alkyl group having 1 to 10 carbon atoms represented by the R⁸ to R¹² may be exemplified by those identical to the examples in the R¹⁰. In addition, the alicyclic structure formed from R¹¹ and R¹² taken together with the carbon atom to which R¹¹ and R¹² bond is exemplified by a cyclopentyl group, a cyclohexyl group, a cycloheptyl group, and the like.

Examples of the group represented by the above formula (7) include a methyl group, an ethyl group, a 1-propyl group, a 2-propyl group, a 1-butyl group, a 2-butyl group, a 1-pentyl group, a 2-pentyl group, a 3-pentyl group, a 1-(2-methylbutyl) group, a 1-(3-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(3-methylpentyl) group, a 2-(4-methylpentyl) group, a 3-(2-methylpentyl) group, and the like. Of these, a methyl group, an ethyl group, a 1-propyl group, a 2-propyl group, a 1-butyl group and a 2-butyl group are preferable.

The base-cleavable bond binds to R² via R³¹¹. The bivalent linking group represented by R³¹¹ may be similarly defined to the bivalent linking group in the acid cleavable bond.

The organic acid ion moiety in the organic acid represented by the above formula (I) is, for example, represented by the following formula:

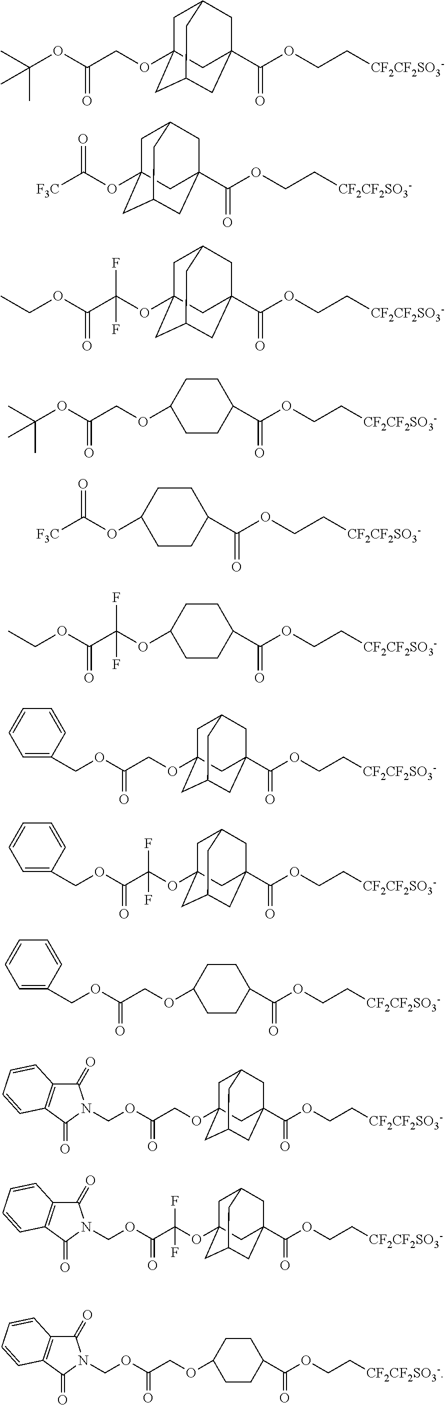

It is to be noted that the organic acid not only forms an acid generating agent together with a cation capable of forming the acid generating agent described later but also may be present in the form of a salt such as an alkali metal salt, an alkaline earth metal salt or an ester, for example. These salts of the organic acid are useful as a precursor of an acid generating agent, and the like.

In a synthesis method of the organic acid, conventionally well-known methods can be employed in combination thereof. For example, a hydroxyl group in hydroxyadamantanecarboxylate is protected; the hydroxyl group in hydroxyadamantanecarboxylate is reacted with a halogenated hydrocarbon in the presence of an alkoxide to generate a halogenated alkyl ester of a carboxylic acid; then, a sulfinic acid group is introduced into a halogenated alkyl moiety under a basic condition; a sulfonic acid group is successively obtained under an oxidization condition; and thereafter, deprotection of the hydroxyl group is finally carried out to prepare a precursor of the organic acid. Next, in the exemplary procedure, the hydroxyl group of the obtained precursor is esterified under a basic condition and finally an onium salt of the sulfonic acid is obtained. However, as a procedure of synthesizing the organic acid, other procedure may be employed as long as the aforementioned organic acid can be obtained.

A counter ion that constitutes an acid generating agent together with the aforementioned organic acid ion is not particularly limited as long as it is a cation capable of stably forming the acid generating agent (A) together with an organic acid ion.

Examples of the cation include onium cations such as O, S, Se, N, P, As, Sb, Cl, Br, I, and the like. Of these, S and I are preferable. In other words, the acid generating agent (A) is preferably a sulfonium salt compound or an iodonium salt compound of the organic acid represented by the above formula (I). When the acid generating agent (A) has a form of the aforementioned salt compound, a deprotection reaction of a sulfo group by a radioactive ray can be facilitated, whereby radiation-sensitivity of the acid generating agent (A) can be enhanced.

A monovalent onium cation is exemplified by a cation represented by the following formula (12) or (13), and the like.

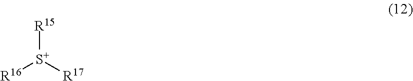

In the above formula (12), R¹⁵, R¹⁶ and R¹⁷ each independently represent an optionally substituted linear or branched alkyl group having 1 to 10 carbon atoms, or an optionally substituted aryl group having 6 to 18 carbon atoms, wherein any two or more of R¹⁵, R¹⁶ and R¹⁷ may taken together represent a ring structure together with the sulfur atom to which they bond.

R¹⁸—I⁺—R¹⁹ (13)

In the above formula (13), R¹⁸ and R¹⁹ each independently represent an optionally substituted linear or branched alkyl group having 1 to 10 carbon atoms, or an optionally substituted aryl group having 6 to 18 carbon atoms, wherein R¹⁸ and R¹⁹ may taken together represent a ring structure together with the iodine atom to which R¹⁸ and R¹⁹ bond.

The Onium cation represented by the above formula (12) is preferably an onium cation represented by the following formulae (12-1) and (12-2), and the onium cation represented by the above formula (13) is preferably an onium cation represented by the following formula (13-1).

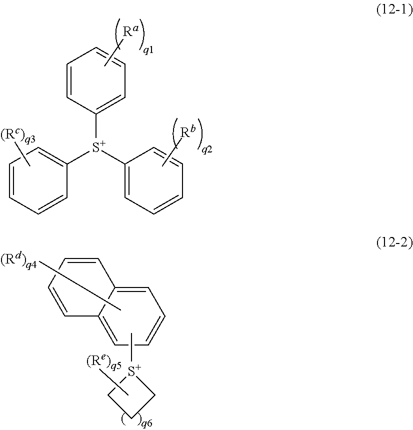

In the above formula (12-1), R^{a}, R^{b} and R^{c} each independently represent a hydrogen atom, a hydroxyl group, a halogen atom, an optionally substituted linear or branched alkyl group having 1 to 12 carbon atoms or an optionally substituted aryl group having 6 to 12 carbon atoms; q1, q2 and q3 are each independently an integer of 0 to 5, wherein provided that R^{a}, R^{b} and R^{c} are present in a plurality of number, the R^{a}, the R^{b} and the R^{c} present in a plurality of number may be the same or different; R^{a}, R^{b} and R^{c} present in the number of at least two may taken together represent a ring structure. In the above formula (12-2), R^{d} represents a hydrogen atom, an optionally substituted linear or branched alkyl group having 1 to 8 carbon atoms, or an optionally substituted aryl group having 6 to 8 carbon atoms; R^{e} represents a hydrogen atom, an optionally substituted linear or branched alkyl group having 1 to 7 carbon atoms, or an optionally substituted aryl group having 6 to 7 carbon atoms; q4 is an integer of 0 to 7; q5 is an integer of 0 to 6; q6 is an integer of 0 to 3, wherein provided that R^{d} and R^{e} are each present in a plurality of number, the R^{d} and R^{e} present in a plurality of number may be the same or different; and R^{d} and R^{e} present in the number of at least two may taken together represent a ring structure.

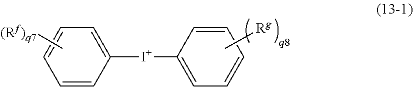

In the above formula (13-1), R^{f} and R^{g} each independently represent a hydrogen atom, a nitro group, a halogen atom, an optionally substituted linear or branched alkyl group having 1 to 12 carbon atoms, or an optionally substituted aryl group having 6 to 12 carbon atoms; q7 and q8 are each independently an integer of 0 to 5, wherein provided that R^{f} and R^{g} are present in a plurality of number, the R^{f} and R^{g} present in a plurality of number may be the same or different; and further, R^{f} and R^{g} present in the number of at least two may taken together represent a ring structure.

The onium cations represented by the above formulae (12-1) and (12-2) are exemplified by onium cations represented by the following formulae (i-1) to (i-64), and the like. The onium cations represented by the above formula (13-1) are exemplified by onium cations represented by the following formulae (ii-1) to (ii-39), and the like.

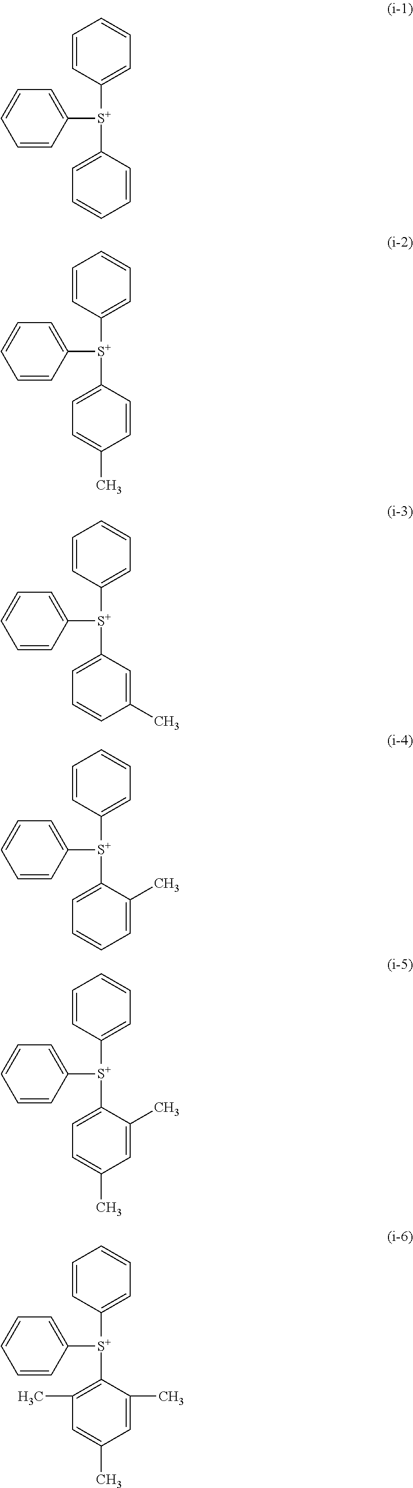 [SMILES: c1ccc([S+](c2ccccc2)c2ccccc2)cc1 ; Cc1ccc([S+](c2ccccc2)c2ccccc2)cc1 ; Cc1cccc([S+](c2ccccc2)c2ccccc2)c1 ; Cc1ccccc1[S+](c1ccccc1)c1ccccc1 ; Cc1ccc([S+](c2ccccc2)c2ccccc2)c(C)c1 ; Cc1cc(C)c([S+](c2ccccc2)c2ccccc2)c(C)c1]

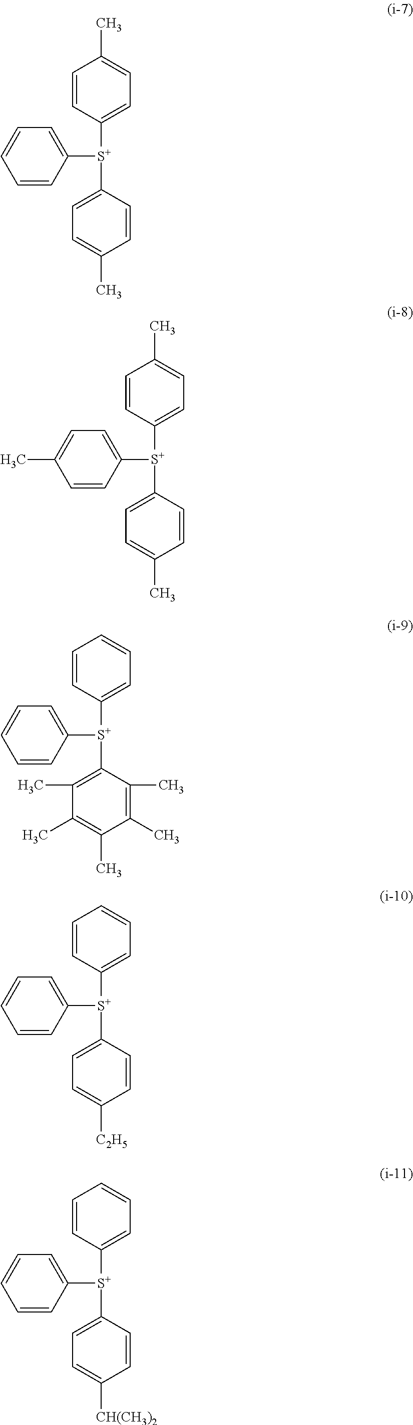 [SMILES: Cc1ccc([S+](c2ccccc2)c2ccc(C)cc2)cc1 ; Cc1ccc([S+](c2ccc(C)cc2)c2ccc(C)cc2)cc1 ; Cc1c(C)c(C)c([S+](c2ccccc2)c2ccccc2)c(C)c1C ; CCc1ccc([S+](c2ccccc2)c2ccccc2)cc1 ; CC(C)c1ccc([S+](c2ccccc2)c2ccccc2)cc1]

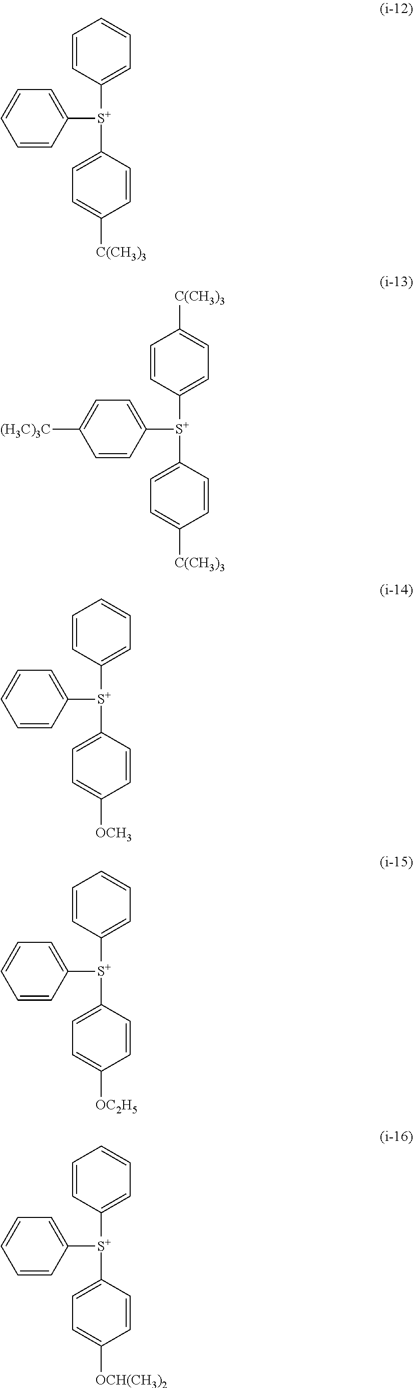

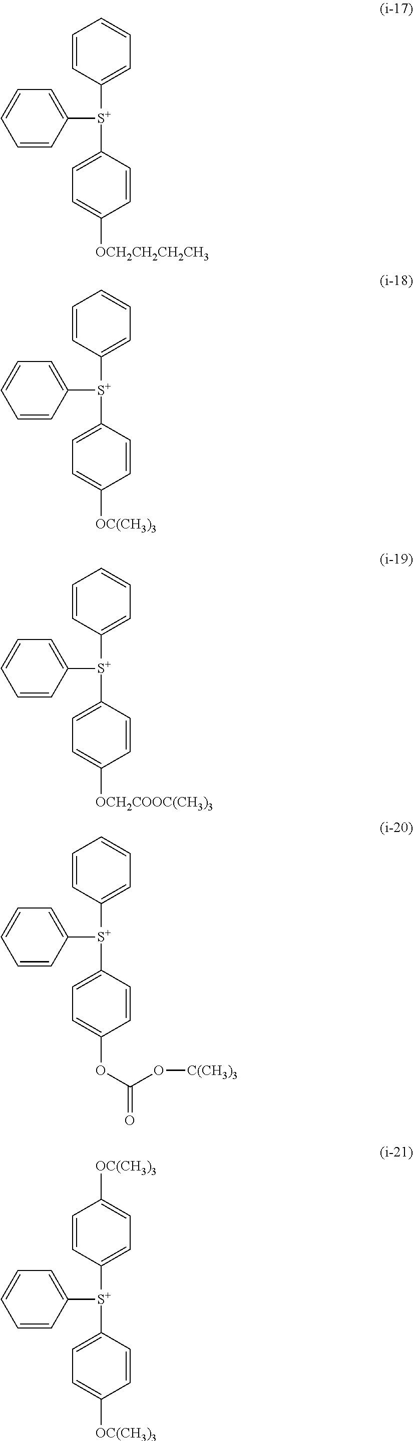

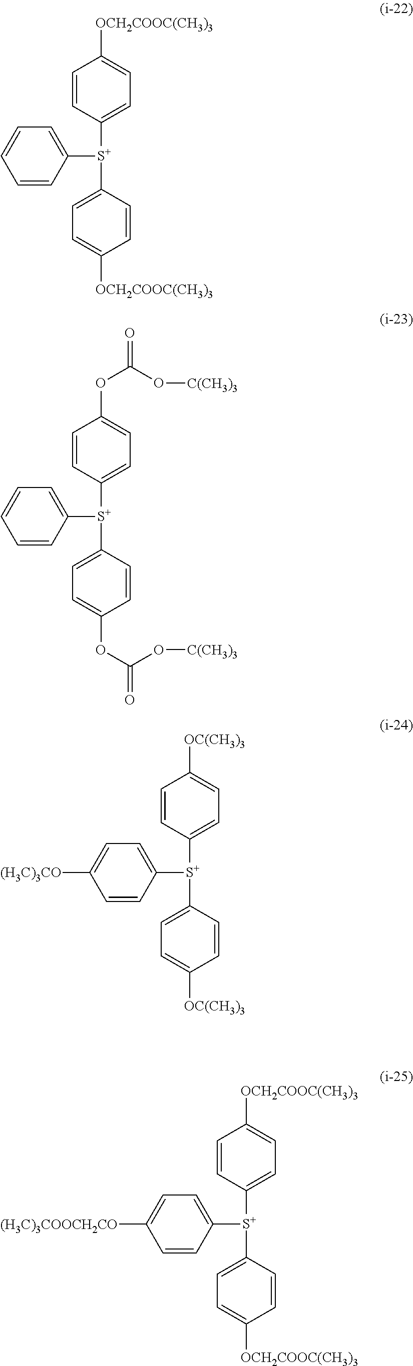

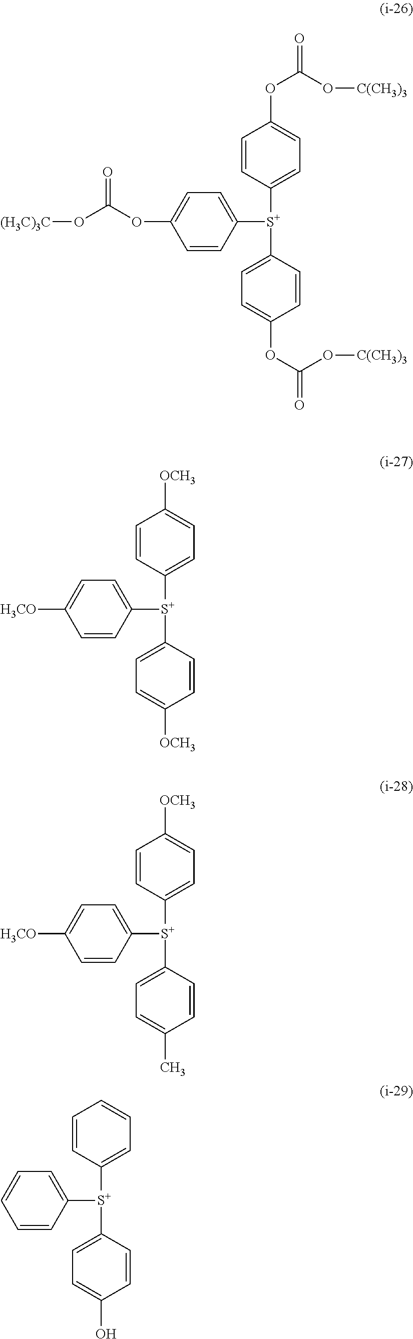 [SMILES: CC(C)(C)OC(=O)Oc1ccc([S+](c2ccc(OC(=O)OC(C)(C)C)cc2)c2ccc(OC(=O)OC(C)(C)C)cc2)cc1 ; COc1ccc([S+](c2ccc(OC)cc2)c2ccc(OC)cc2)cc1 ; COc1ccc([S+](c2ccc(C)cc2)c2ccc(OC)cc2)cc1 ; Oc1ccc([S+](c2ccccc2)c2ccccc2)cc1]

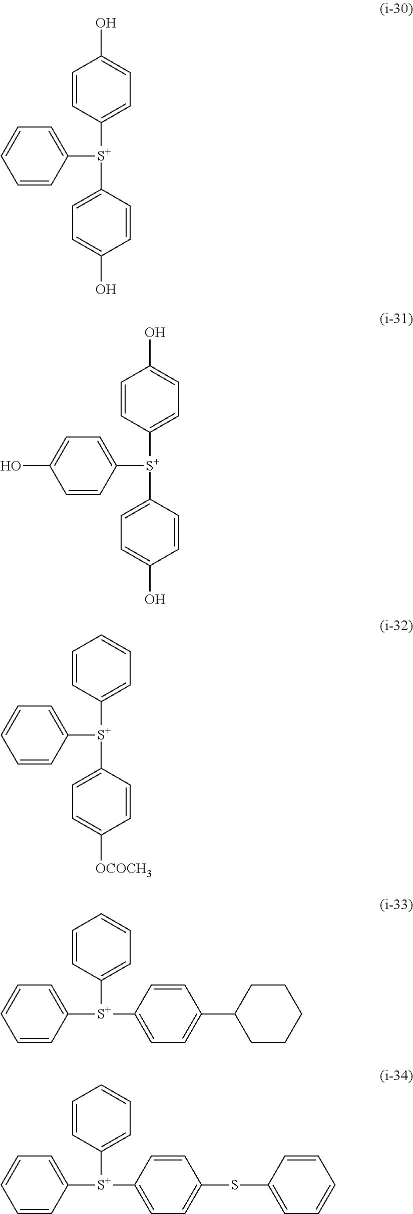

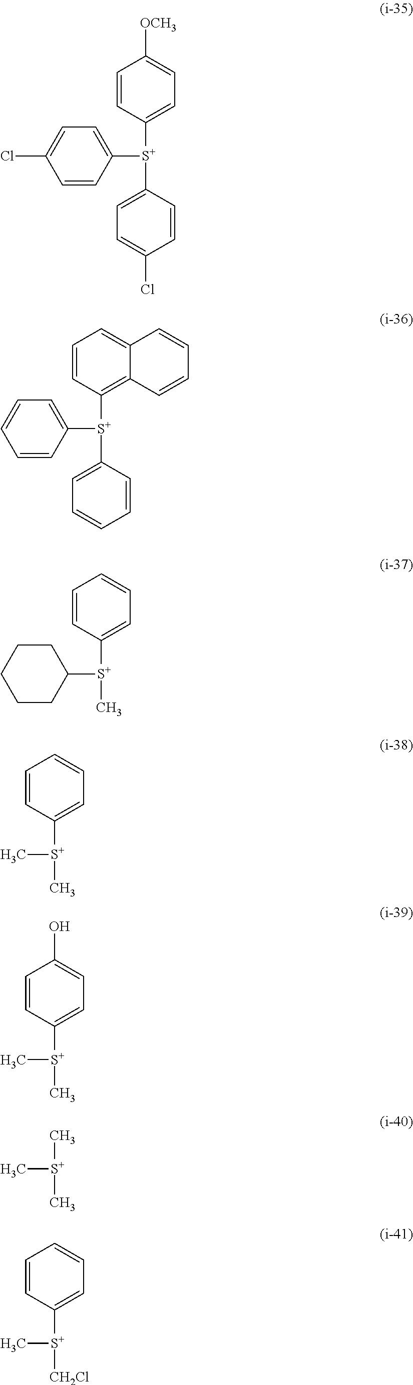

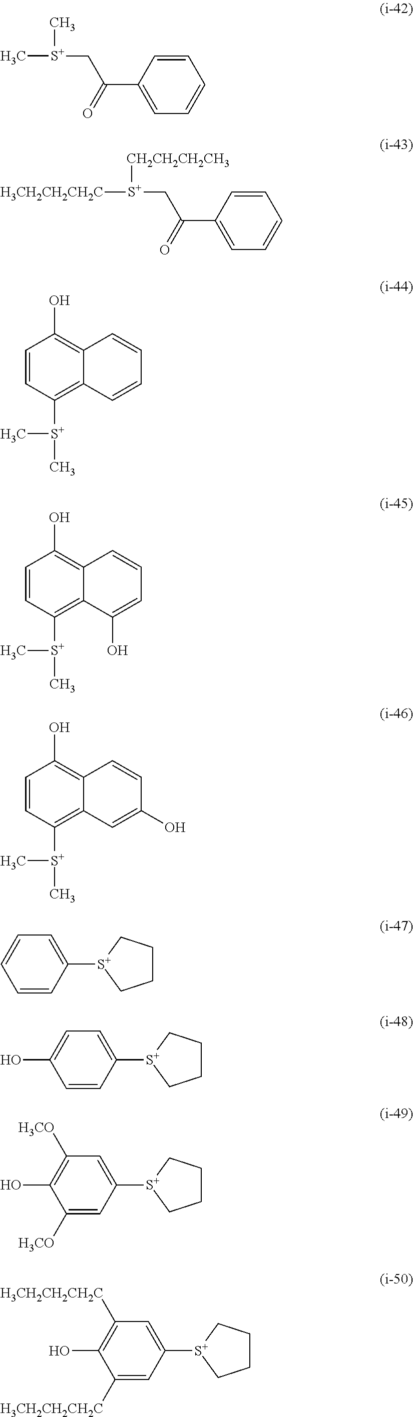

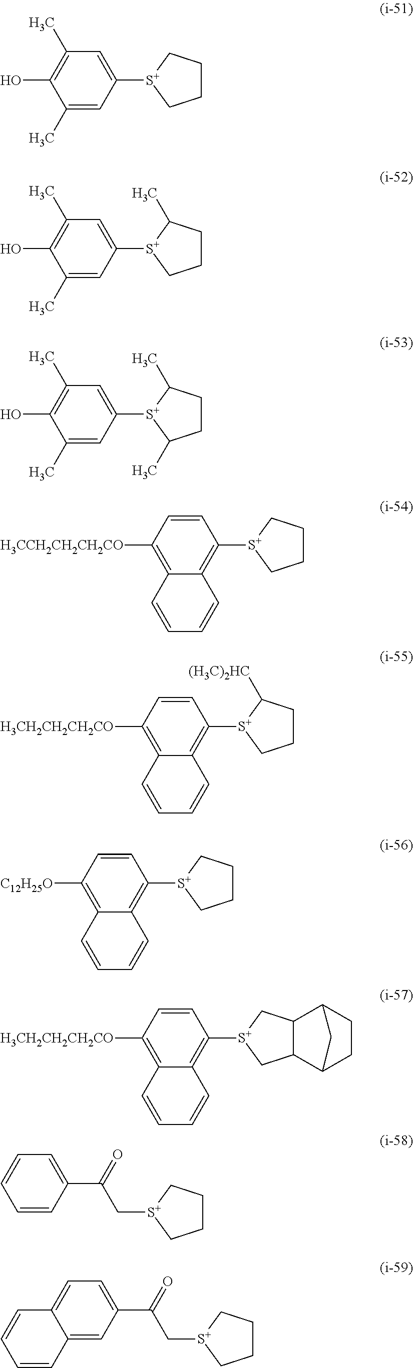

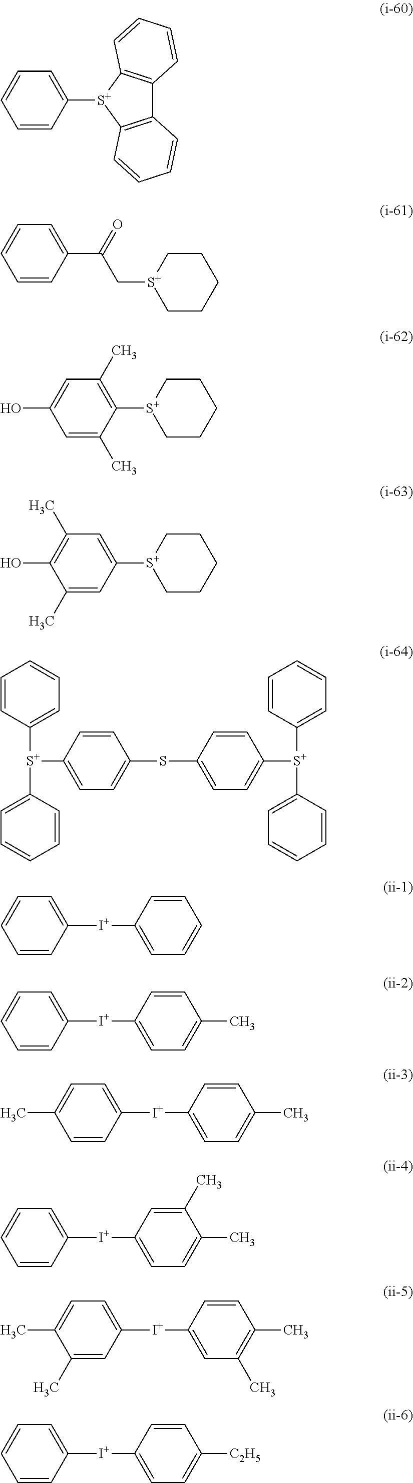

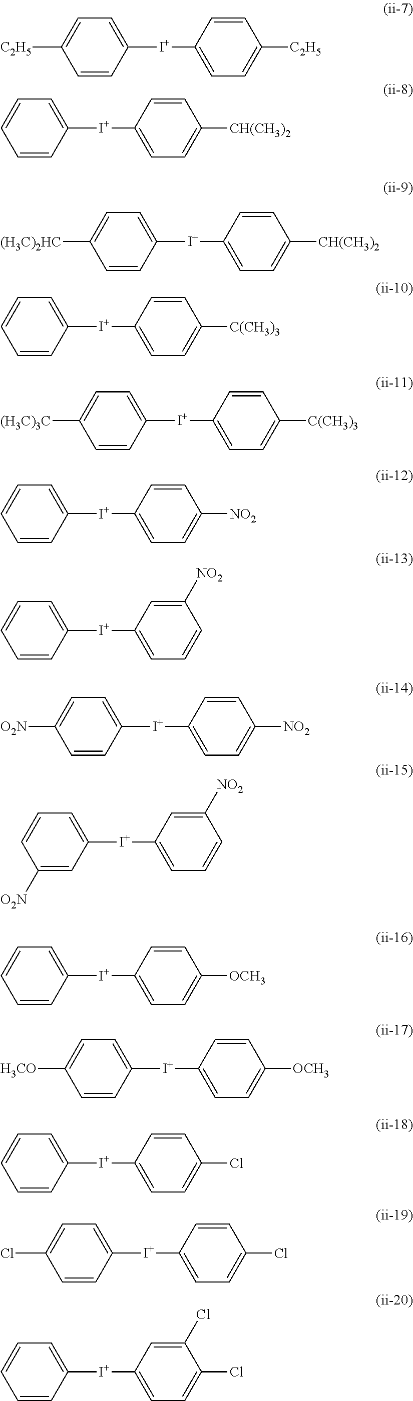

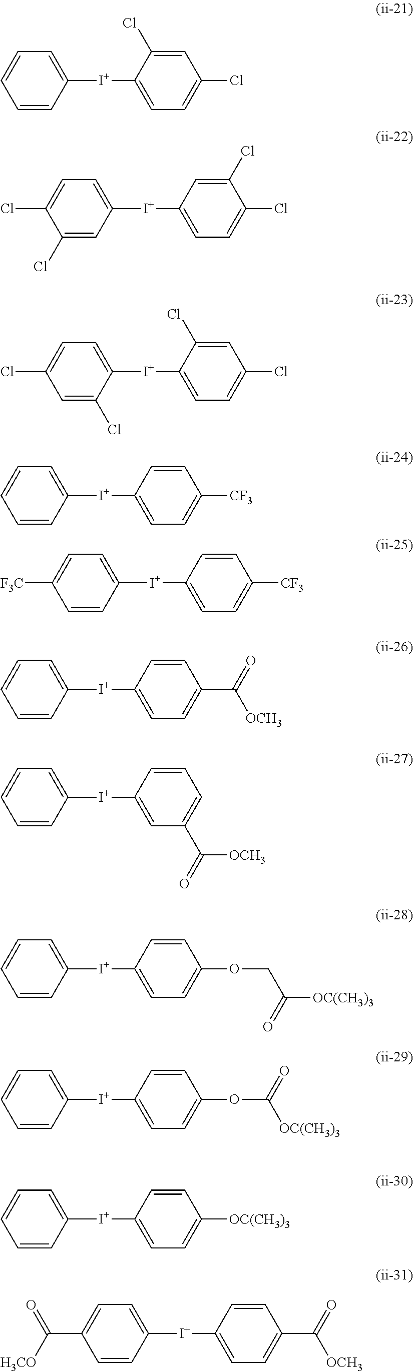

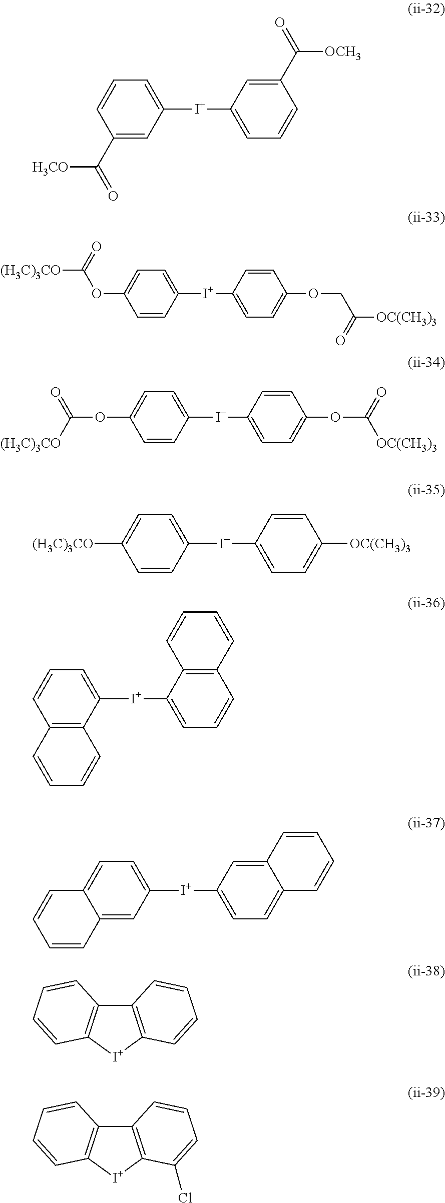

Of these, cations represented by the above formula (i-1), formula (i-2), formula (i-6), formula (i-8), formula (i-13), formula (i-19), formula (i-25), formula (i-27), formula (i-29), formula (i-33), formula (i-51), formula (i-54), formula (ii-1) and formula (ii-11) are more preferable.

The monovalent onium cation described above can be produced according to a general method described in, for example, Advances in Polymer Science, Vol. 62, p. 1-48 (1984).

The content of the acid generating agent (A) may be determined depending on the type of the polymer contained in the radiation-sensitive resin composition and is preferably 0.1 parts by mass to 30 parts by mass, more preferably 2 parts by mass to 27 parts by mass, and particularly preferably 5 parts by mass to 25 parts by mass with respect to 100 parts by mass of (B) a polymer. When the content of the acid generating agent (A) is less than 0.1 parts by mass, sensitivity and/or resolution as a photoresist film may be deteriorated. To the contrary, when the content of the acid generating agent (A) exceeds 30 parts by mass, coating properties and pattern configuration as a photoresist film may be deteriorated.

### <(B) Polymer>

The composition preferably contains (B) a polymer. The polymer (B) serves as a base resin of the composition. The polymer is exemplified by a polymer which is insoluble or hardly soluble in alkali and has an acid-dissociable group and which becomes easily soluble in alkali when the acid-dissociable group dissociates (hereinafter, may also be referred to as “(B1) acid-dissociable group-containing polymer”) and a polymer which has one or more types of functional group(s) that exhibits an affinity to an alkaline developer typified by an oxygen-containing functional group such as a phenolic hydroxyl group, an alcoholic hydroxyl group or a carboxyl group and which is soluble in an alkaline developer (hereinafter, may also be referred to as “(B2) alkali-soluble polymer”). A radiation-sensitive resin composition including the polymer (B1) can be suitably used as a positive type radiation-sensitive resin composition, whereas a radiation-sensitive resin composition including the polymer (B2) can be suitably used as a negative type radiation-sensitive resin composition.

In the case in which the polymer (B) is used together with (C) a fluorine atom-containing polymer described later, the proportion of fluorine atom(s) in the polymer (B) contained is preferably smaller than that of the fluorine atom-containing polymer (C). The proportion of fluorine atom(s) in the polymer (B) contained is typically less than 10% by mass, preferably 0% by mass to 9% by mass and more preferably 0% by mass to 6% by mass with respect to 100% by mass of the total of the polymer (B). It is to be noted that herein the proportion of fluorine atom(s) contained can be determined by ¹³C-NMR.

In the case in which a photoresist film is formed using a radiation-sensitive resin composition including the polymer (B) and the fluorine atom-containing polymer (C), the distribution of the fluorine atom-containing polymer (C) on the surface of the photoresist film tends to be high due to hydrophobicity of the fluorine atom-containing polymer (C). In other words, the fluorine atom-containing polymer (C) unevenly distributes on the surface layer of the photoresist film. Therefore, there is no need to separately form an upper layer film for the purpose of blocking a photoresist film from a liquid for immersion lithography, and thus the radiation-sensitive resin composition including the polymer (B) and the fluorine atom-containing polymer (C) can be suitably used in a liquid immersion lithography process.

### [(B1) Acid-Dissociable Group-Containing Polymer]

The acid-dissociable group-containing polymer (B1) is a polymer having an acid-dissociable group in a main chain, a side chain or both the main chain and the side chain. Of these, a polymer having an acid-dissociable group in the side chain is preferable.

The acid-dissociable group-containing polymer (B1) includes a structural unit having an acid-dissociable group (hereinafter, may be also referred to as “structural unit (b1)”). In addition, the acid-dissociable group-containing polymer (B1) may include a structural unit having a lactone skeleton (hereinafter, may be also referred to as “structural unit (b2)”) and other structural units. Hereinafter, each structural unit will be explained in detail.

(Structural Unit (b1))

The structural unit (b1) is exemplified by a structural unit represented by the following formula (14), and the like.

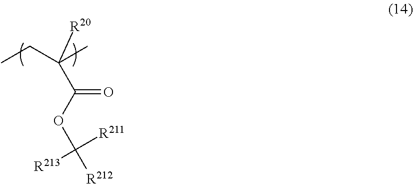

In the above formula (14), R²⁰ represents a hydrogen atom, a methyl group, a trifluoromethyl group or a hydroxymethyl group. R²¹¹, R²¹² and R²¹³ are as defined in connection with R⁵, R⁶ and R⁷ in the above formula (2).

The structural unit (b1) is preferably a structural unit represented by the following formula (14-1).

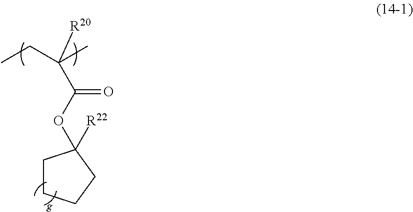

In the above formula (14-1), R²⁰ is as defined in connection with the formula (14); R²² represents a linear or branched alkyl group having 1 to 4 carbon atoms; and g is an integer of 1 to 4.

Examples of the alkyl group having 1 to 4 carbon atoms represented by the R²² include a methyl group, an ethyl group, an n-propyl group, an isopropyl group, an n-butyl group, a 2-methylpropyl group, a 1-methylpropyl group, a tert-butyl group, and the like.

The structural unit (b1) may be used either alone or two or more types in combination thereof. A monomer that gives the structural unit (b1) is preferably (meth)acrylic acid 2-methyl-2-cyclopentyl ester, (meth)acrylic acid 2-ethyl-2-cyclopentyl ester, (meth)acrylic acid 2-isopropyl-2-cyclopentyl ester, (meth)acrylic acid 2-methyl-2-cyclohexyl ester, (meth)acrylic acid 2-ethyl-2-cyclohexyl ester and (meth)acrylic acid 2-ethyl-2-cyclooctyl ester.

The proportion of the structural unit (b1) in the acid-dissociable group-containing polymer (B1) contained is preferably 5 mol % to 85 mol %, more preferably 10 mol % to 70 mol % and particularly preferably 15 mol % to 60 mol % with respect to the total of the structural unit. When the proportion of the structural unit (b1) contained is less than 5 mol %, developability and exposure latitude may be deteriorated. To the contrary, when the proportion of the structural unit (b1) contained exceeds 85 mol %, solubility of the acid-dissociable group-containing polymer (B1) into a solvent may be deteriorated or resolution may be deteriorated.

(Structural Unit (b2))

The structural unit (b2) is exemplified by structural units represented by the following formulae (17-1) to (17-6), and the like.

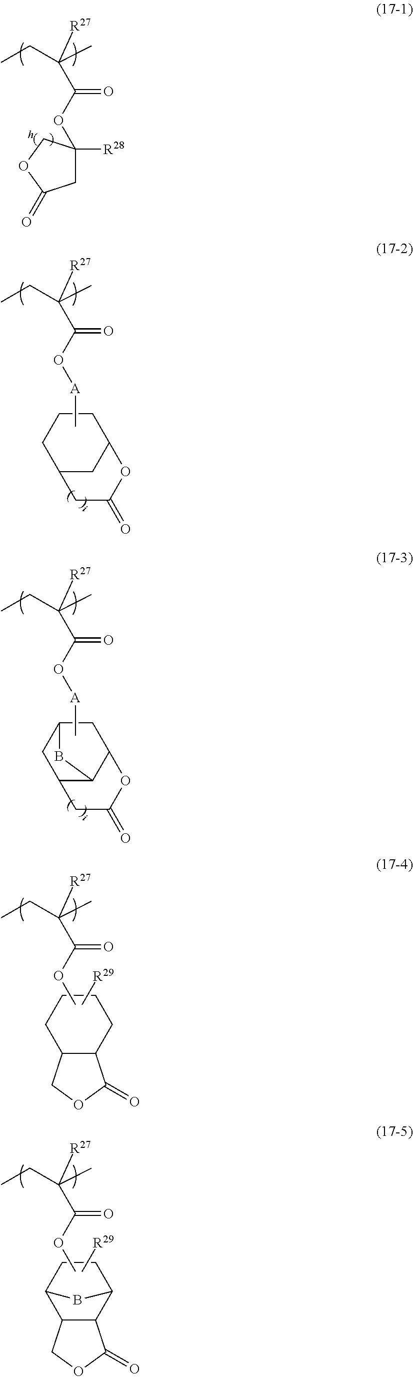

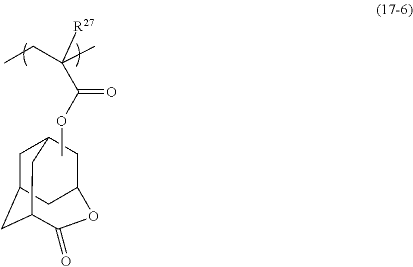

In the above formula, R²⁷ each independently represents a hydrogen atom or a methyl group; R²⁸ represents a hydrogen atom or an optionally substituted alkyl group having 1 to 4 carbon atoms; R²⁹ represents a hydrogen atom or a methoxy group; A represents a single bond or a bivalent linking group; B represents an oxygen atom or a methylene group; h is an integer of 1 to 3; and i is 0 or 1.

Examples of the alkyl group having 1 to 4 carbon atoms represented by the R²⁸ include a methyl group, an ethyl group, an n-propyl group, an isopropyl group, an n-butyl group, a 2-methylpropyl group, a 1-methylpropyl group, a tert-butyl group, and the like. Examples of the substituent of the optionally substituted alkyl group having 1 to 4 carbon atoms represented by R²⁸ include a halogen atom (fluorine atom, chlorine atom, bromine atom, etc.), a phenyl group, an acetoxy group, an alkoxy group, and the like. The R³¹ may be similarly defined to the bivalent linking group represented by the A.

In the case in which the acid-dissociable group-containing polymer (B1) includes the structural unit (b2), the proportion of the structural unit (b2) contained is preferably 10 mol % to 70 mol %, more preferably 15 mol % to 60 mol % and particularly preferably 20 mol % to 50 mol % with respect to the total of the structural unit. When the proportion of the structural unit (b2) contained is less than 10 mol %, resolution as a resist may be decreased. To the contrary, when the proportion of the structural unit (b2) contained exceeds 70 mol %, developability and exposure latitude may be deteriorated.

### (Other Structural Unit)

The acid-dissociable group-containing polymer (B1) may include other structural unit except for the structural unit (b1) and the structural unit (b2). Examples of the other structural unit include structural units having a hydroxyalkyl(meth)acrylate such as 2-hydroxyethyl(meth)acrylate, 2-hydroxypropyl(meth)acrylate or 3-hydroxypropyl(meth)acrylate; structural units having alkali-solubility described later; structural units having a cyclic carbonate structure; structural units having an alicyclic structure described in WO2007/116664, and the like.

### [Synthesis Method of (B1) Acid-Dissociable Group-Containing Polymer]

The acid-dissociable group-containing polymer (B1) can be synthesized by polymerizing a monomer that gives the structural unit (b1), for example, in the presence of a chain transfer agent in a solvent to which a radical polymerization initiator (hydroperoxide, dialkylperoxide, diacylperoxide, an azo compound, etc.) was added.

Examples of the solvent include alkanes such as n-pentane, n-hexane, n-heptane, n-octane, n-nonane and n-decane; cycloalkanes such as cyclohexane, cycloheptane and cyclooctane; alicyclic hydrocarbons such as decalin and norbornane; aromatic hydrocarbons such as benzene, toluene, xylene, ethylbenzene and cumene; halogenated hydrocarbons such as chlorobutane, bromohexane, dichloroethane, hexamethylenedibromide and chlorobenzene; saturated carboxylate esters such as ethyl acetate, n-butyl acetate, isobutyl acetate, methyl propionate; ketones such as acetone, 2-butanone, 4-methyl-2-pentanone, 2-heptanone; ethers such as tetrahydrofuran, dimethoxyethane and diethoxyethane; alcohols such as methanol, ethanol, 1-propanol, 2-propanol and 4-methyl-2-pentanol, and the like. It is to be noted that these solvents may be used either alone or two or more types in combination thereof.

The polymerization temperature is preferably 40° C. to 150° C. and more preferably 50° C. to 120° C. The reaction time is preferably 1 hour to 48 hrs and more preferably 1 hour to 24 hrs. It is to be noted that the content of impurities such as halogens and metals in the acid-dissociable group-containing polymer (B1) is more favorably as low as possible. When the content of impurities is lower, sensitivity, resolution, process stability, pattern configuration, and the like of a photoresist film can be further enhanced. Therefore, a purification method of the acid-dissociable group-containing polymer (B1) is exemplified by chemical purification methods such as washing with water and liquid-liquid extraction, combination methods of the chemical purification methods with physical purification methods such as ultrafiltration and centrifugal separation, and the like.

Examples of the solvent used for liquid-liquid extraction include alkanes such as n-pentane, n-hexane, n-heptane, n-octane, n-nonane and n-decane; alcohols such as methanol, ethanol, 1-propanol, 2-propanol and 4-methyl-2-pentanol; ketones such as acetone, 2-butanone, 4-methyl-2-pentanone and 2-heptanone. Of these, n-hexane, n-heptane, methanol, ethanol, acetone and 2-butanone are preferable.

The weight average molecular weight (Mw) of the acid-dissociable group-containing polymer (B1) is preferably 1,000 to 50,000, more preferably 1,000 to 40,000, particularly preferably 1,000 to 30,000 in terms of the polystyrene equivalent on gel permeation chromatography (GPC). When the Mw is less than 1,000, a photoresist film having a sufficient receding contact angle may not be obtained. To the contrary, when the Mw exceeds 50,000, developability in a photoresist film may be decreased.

The ratio (Mw/Mn) of the number average molecular weight (Mn) in terms of the polystyrene equivalent on GPC to the Mw is preferably 1 to 5 and more preferably 1 to 4.

### [(B2) Alkali-Soluble Polymer]

The alkali-soluble polymer (B2) is exemplified by a polymer prepared by addition polymerization having at least one type structural unit selected from the group consisting of structural units represented by the following formulae, respectively, and the like. Hereinafter, each structural unit is referred to as (B2-1) a structural unit, (B2-2) a structural unit and (B2-3) a structural unit, respectively.

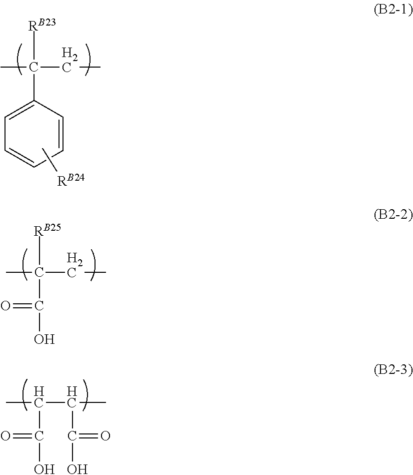

In the above formula (B2-1) and formula (B2-2), R^{B23} and R^{B25} each independently represent a hydrogen atom or a methyl group; R^{B24} represents a hydroxyl group, a carboxyl group, —R^{B26}COOH, —OR^{B26}COOH, —OCOR^{B26}COOH or COOR^{B26}COOH; R^{B26} represents —(CH₂)ₘ—; and m is an integer of 1 to 4.

The alkali-soluble polymer (B2) may be configured with only the structural unit (B2-1), the structural unit (B2-2) or the structural unit (B2-3), but one type or more other structural units may be included as long as a synthesized polymer is soluble in an alkaline developer. The other structural units are exemplified by a structural unit similar to other structural unit in the acid-dissociable group-containing polymer (B1), and the like.

The proportion of the total of the structural unit (B2-1), the structural unit (B2-2) and the structural unit (B2-3) contained in the alkali-soluble polymer (B2) is preferably 10 mol % to 100 mol % and more preferably 20 mol % to 100 mol %.

When the alkali-soluble polymer (B2) has a structural unit having a carbon-carbon unsaturated bond like the structural unit (B2-1), the alkali-soluble polymer (B2) can be also used as a hydrogenated product. In such a case, the hydrogenation rate is typically no greater than 70%, preferably no greater than 50% and more preferably no greater than 40% of a carbon-carbon unsaturated bond included in the structural unit. When the hydrogenation rate exceeds 70%, alkali developability of the alkali-soluble polymer (B2) may be decreased.

The alkali-soluble polymer (B2) is preferably a polymer having poly(4-hydroxystyrene), a 4-hydroxystyrene/4-hydroxy-α-methylstyrene copolymer or a 4-hydroxystyrene/styrene copolymer as a principal component.

The Mw of the alkali-soluble polymer (B2) is typically 1,000 to 150,000 and preferably 3,000 to 100,000. In the composition, the alkali-soluble polymer (B2) may be used either alone or two or more types in combination thereof.

### <(C) Fluorine Atom-Containing Polymer>

The fluorine atom-containing polymer (C) capable of being suitably contained in the composition is a polymer having fluorine atom(s) in a main chain, a side chain, or a main chain and a side chain thereof. Due to the fluorine atom-containing polymer (C), a layer having water repellency is formed in the vicinity of the surface of the photoresist film. Therefore, elution of the acid generating agent, an acid diffusion control agent or the like into a liquid for immersion lithography is prevented. In addition, due to an increase in a receding contact angle between the photoresist film and a liquid for immersion lithography, water droplets derived from the liquid for immersion lithography are less likely to remain on the photoresist film, thereby resulting in prevention defects caused by the liquid for immersion lithography from occurring.

The fluorine atom-containing polymer (C) preferably has a structural unit having a fluorine atom (hereinafter, may be also referred to as structural unit (c1)).

[Structural Unit (c1)]

The structural unit (c1) is not particularly limited as long as the structural unit (c1) has fluorine atom(s) and preferably contains structural units represented by the following formulae (c1-1) to (c1-3). Hereinafter, each structural unit is referred to as (c1-1) a structural unit, (c1-2) a structural unit and (c1-3) a structural unit.

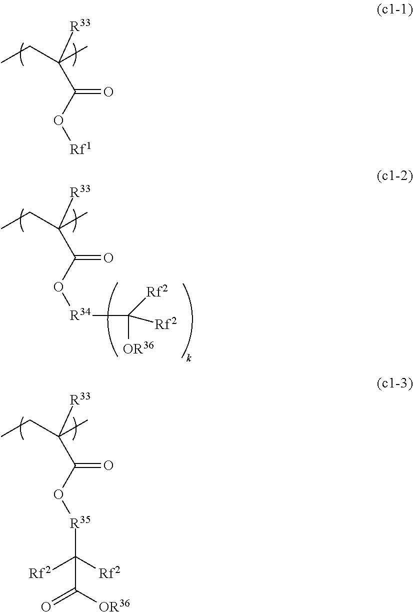

In the above formulae (c1-1) to (c1-3), R³³ each independently represents a hydrogen atom, a lower alkyl group or a halogenated lower alkyl group. In the formula (c1-1), Rf¹ represents a fluorinated alkyl group having 1 to 30 carbon atoms; R³⁴ represents a linking group having a valency of (k+1); R³⁶ represents a monovalent organic group including a hydrogen atom, an acid-dissociable group or a base-dissociable group; k is an integer of 1 to 3; Rf² each independently represents a hydrogen atom, a fluorine atom or a fluorinated alkyl group having 1 to 30 carbon atoms, wherein provided that Rf² and R³⁶ are present in a plurality of number, the Rf² and R³⁶ present in a plurality of number may be each the same or different; but any case where all the Rf² are a hydrogen atom is excluded; and R³⁵ represents a bivalent linking group.

The fluorinated alkyl group having 1 to 30 carbon atoms represented by the Rf¹ is exemplified by a linear or branched alkyl group having 1 to 6 carbon atoms substituted with at least one or more fluorine atom(s), a monovalent alicyclic hydrocarbon group having 4 to 20 carbon atoms substituted with at least one or more fluorine atom(s) or a group derived therefrom, and the like.

Examples of the linear or branched alkyl group having 1 to 6 carbon atoms include a methyl group, an ethyl group, a 1-propyl group, a 2-propyl group, a 1-butyl group, a 2-butyl group, a 2-(2-methylpropyl) group, a 1-pentyl group, a 2-pentyl group, a 3-pentyl group, a 1-(2-methylbutyl) group, a 1-(3-methylbutyl) group, a 2-(2-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(2-methylpentyl) group, a 2-(3-methylpentyl) group, a 2-(4-methylpentyl) group, a 3-(2-methylpentyl) group, a 3-(3-methylpentyl) group, and the like.

Examples of the monovalent alicyclic hydrocarbon group having 4 to 20 carbon atoms or a group derived therefrom include a cyclopentyl group, a cyclopentylmethyl group, a 1-(1-cyclopentylethyl) group, a 1-(2-cyclopentylethyl) group, a cyclohexyl group, a cyclohexylmethyl group, a 1-(1-cyclohexylethyl) group, a 1-(2-cyclohexylethyl) group, a cycloheptyl group, a cycloheptylmethyl group, a 1-(1-cycloheptylethyl) group, a 1-(2-cycloheptylethyl) group, and the like.

Preferable examples of the monomer that gives the structural unit (c1-1) include trifluoromethyl(meth)acrylic acid esters, 2,2,2-trifluoroethyl(meth)acrylic acid esters, perfluoroethyl(meth)acrylic acid esters, perfluoro n-propyl(meth)acrylic acid esters, perfluoro i-propyl(meth)acrylic acid esters, perfluoro n-butyl(meth)acrylic acid esters, perfluoro i-butyl(meth)acrylic acid esters, perfluoro t-butyl(meth)acrylic acid esters, 2-(1,1,1,3,3,3-hexafluoropropyl)(meth)acrylic acid esters, 1-(2,2,3,3,4,4,5,5-octafluoropentyl)(meth)acrylic acid esters, perfluorocyclohexylmethyl(meth)acrylic acid esters, 1-(2,2,3,3,3-pentafluoropropyl)(meth)acrylic acid esters, 1-(3,3,4,4,5,5,6,6,7,7,8,8,9,9,10,10,10-heptadecafluorodecyl)(meth)acrylic acid esters, 1-(5-trifluoromethyl-3,3,4,4,5,6,6,6-octafluorohexyl)(meth)acrylic acid esters.

The monovalent organic group represented by the R³⁶ is exemplified by a monovalent hydrocarbon group having 1 to 30 carbon atoms, an acid-dissociable group and a base-dissociable group.

The monovalent hydrocarbon group having 1 to 30 carbon atoms represented by the R³⁶ may be similarly defined to, for example, the alkyl group having 1 to 30 carbon atoms represented by the R^{S1} described above.

The acid-dissociable group in the structural unit (c1-2) represented by the R³⁶ is preferably a group represented by the —CR⁵R⁶R⁷ described above, a t-butoxycarbonyl group, an alkoxy-substituted methyl group and more preferably a t-butoxycarbonyl group and an alkoxy-substituted methyl group. The acid-dissociable group represented by the R³⁶ in the structural unit (c1-3) is preferably an alkoxy-substituted methyl group and a group represented by —CR⁵R⁶R⁷ in the above formula (2).

Use of the structural unit having an acid-dissociable group as the structural unit (c1-2) and/or the structural unit (c1-3) is preferable in that solubility of the fluorine atom-containing polymer (C) at patterning light-exposed sites can be enhanced. This advantage is considered to result from generation of a polar group by a reaction of the acid-dissociable group with an acid generated at sites exposed with the light on the photoresist film in an exposure step in a method for forming a resist pattern described later.

The base-dissociable group in the above formula (c1-2) is exemplified by a group represented by the following formula (19-1), and the like.

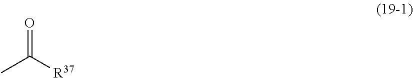

In the above formula (19-1), R³⁷ represents a hydrocarbon group having 1 to 10 carbon atoms and having at least one fluorine atom. The R³⁷ may be similarly defined to the Rf¹ above.

R³⁷ preferably represents a linear or branched perfluoroalkyl group having 1 to 10 carbon atoms obtained by substituting all of hydrogen atoms of the aforementioned hydrocarbon group by a fluorine atom and more preferably a trifluoromethyl group.

The base-dissociable group in the above formula (c1-3) is exemplified by a group represented by the following formulae (19-2) to (19-4), and the like.

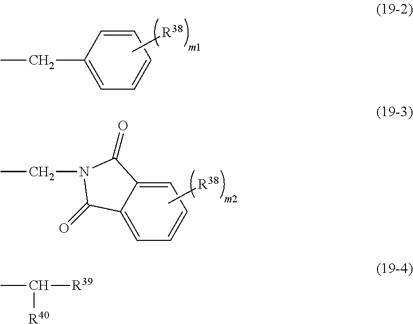

In the above formulae (19-2) and (19-3), R³⁸ represents a halogen atom, an alkoxy group, an acyl group, an acyloxy group or an alkyl group having 1 to 10 carbon atoms; m₁ is an integer of 0 to 5; m₂ is an integer of 0 to 4, wherein provided that R³⁸ is present in a plurality of number, the R³⁸s present in a plurality of number may be the same or different. In the above formula (19-4), R³⁹ and R⁴⁰ each independently represent a hydrogen atom or an alkyl group having 1 to 10 carbon atoms, wherein R³⁹ and R⁴⁰ may taken together represent an alicyclic structure having 4 to 20 carbon atoms together with the carbon atom to which R³⁹ and R⁴⁰ bond.

Examples of the group represented by the formula (19-4) include a methyl group, an ethyl group, a 1-propyl group, a 2-propyl group, a 1-butyl group, a 2-butyl group, a 1-pentyl group, a 2-pentyl group, a 3-pentyl group, a 1-(2-methylbutyl) group, a 1-(3-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(3-methylpentyl) group, a 2-(4-methylpentyl) group, a 3-(2-methylpentyl) group, and the like. Of these, a methyl group, an ethyl group, a 1-propyl group, a 2-propyl group, a 1-butyl group and a 2-butyl group are preferable.

Inclusion of the structural unit having a base-dissociable group in the structural unit (c1-2) and/or the structural unit (c1-3) in the fluorine atom-containing polymer (C) is favorable in that an affinity of the fluorine atom-containing polymer (C) to a developer solution can be enhanced. This advantage is considered to result from generation of a polar group by a reaction of the fluorine atom-containing polymer (C) with a developer solution in a development step in a pattern formation method described later.

In the formulae (c1-2) and (c1-3), in the case in which R³⁶ represents a hydrogen atom, the structural units (c1-2) and (c1-3) have a polar group such as a hydroxyl group or a carboxyl group. Inclusion of such a structural unit in the fluorine atom-containing polymer (C) enables an affinity of the fluorine atom-containing polymer (C) to a developer solution in a development step in a pattern formation method described later to be enhanced.

The linking group having a valency of (k+1) represented by the R³⁴ is exemplified by a single bond, a hydrocarbon group having 1 to 30 carbon atoms and having a valency of (k+1), a group obtained by combining these hydrocarbon groups with a sulfur atom, an imino group, a carbonyl group, —CO—O—, —CO—NH— or the like, and the like.

The R³⁴ having a chain structure is exemplified by a hydrocarbon group having a valency of (k+1) and having a structure obtained by removing (k+1) hydrogen atoms from a chain hydrocarbon having 1 to 10 carbon atoms such as methane, ethane, propane, butane, 2-methylpropane, pentane, 2-methylbutane, 2,2-dimethylpropane, hexane, heptane, octane, nonane or decane, and the like.

The R³⁴ having a cyclic structure is exemplified by a hydrocarbon group having a valency of (k+1) and having a structure obtained by removing (k+1) hydrogen atoms from an alicyclic hydrocarbon having 4 to 20 carbon atoms such as cyclobutane, cyclopentane, cyclohexane, bicyclo[2.2.1]heptane, bicyclo[2.2.2]octane, tricyclo[5.2.1.0^{2,6}]decane or tricyclo[3.3.1.1^{3,7}]decane; a hydrocarbon group having a valency of (k+1) obtained by removing (k+1) hydrogen atoms from an aromatic hydrocarbon having 6 to 30 carbon atoms such as benzene or naphthalene.

The R³⁴ having an oxygen atom, a sulfur atom, an imino group, a carbonyl group, —CO—O— or —CO—NH— is exemplified by groups represented by the following formulae, and the like.

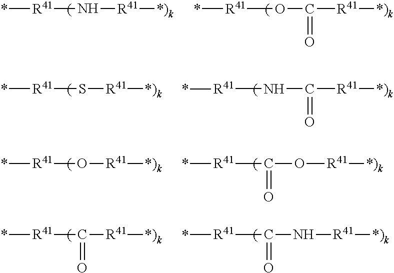

In the above formula, R⁴¹ each independently represents a single bond, a bivalent chain hydrocarbon group having 1 to 10 carbon atoms, a bivalent cyclic hydrocarbon group having 4 to 20 carbon atoms, or a bivalent aromatic hydrocarbon group having 6 to 30 carbon atoms. Examples of these groups represented by R⁴¹ are similar to those in the explanation of the R³⁴ described above.

The R³⁴ may have a substituent. Examples of the substituent include a hydroxy group, a cyano group, and the like.

The bivalent linking group represented by the R³⁵ may be similarly defined to the R³⁴ described above in the case in which k is 1.

The fluorinated hydrocarbon group represented by the Rf² having 1 to 30 carbon atoms may be similarly defined to the Rf¹ described above.

In the formulae (c1-2) and (c1-3), a partial structure represented by the following formula is exemplified by groups represented by the following formulae (f1) to (f5), and the like.

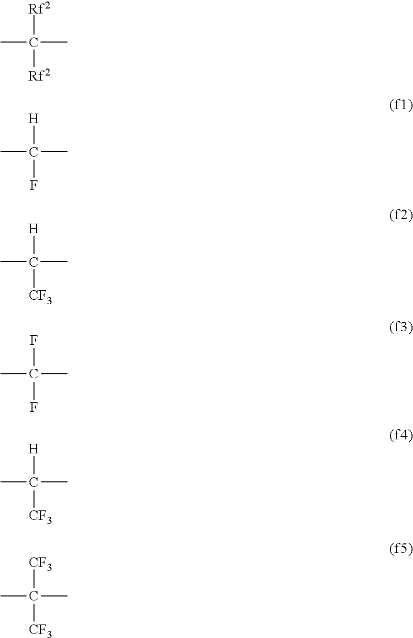

Of these, in the formula (c1-2), a group represented by the above formula (f5) is preferable. In the formula (c1-3), a group represented by the above formula (f3) is preferable.

The structural unit (c1-2) is exemplified by a structural unit represented by the following formulae (c1-2-1) and (c1-2-2), and the like.

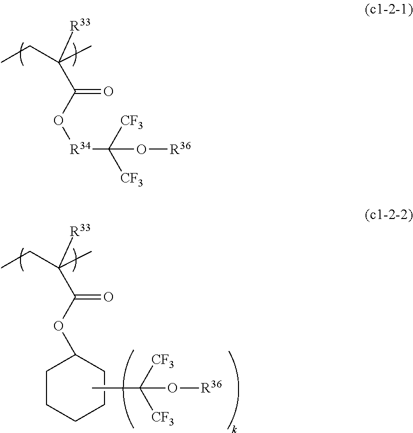

In the above formulae (c1-2-1) and (c1-2-2), R³³, R³⁴, R³⁶ and k are as defined in connection with the above formula (c1-2).

The compound that gives the structural units represented by the above formulae (c1-2-1) and (c1-2-2) is exemplified by compounds represented by the following formulae, and the like.

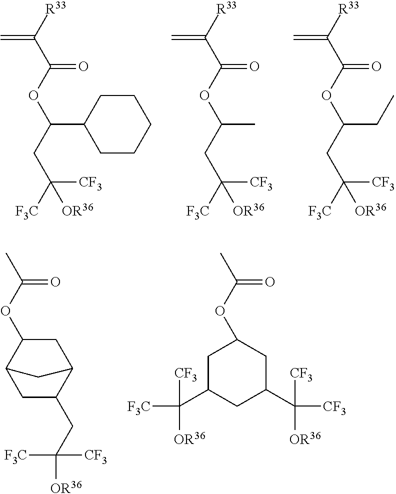

In the above formula, R³³ and R³⁶ are as defined in connection with the above formula (c1-2).

A compound having the acid-dissociable group or base-dissociable group represented by R³⁶ in the above formulae can be synthesized, for example, using as a raw material a compound having a hydrogen atom represented by R³⁶ in the each formula. By way of example, with respect to a compound having a group represented by the above formula (19-1) as R³⁶, the intended compound can be formed by fluoroacylating a compound in which R³⁶ represents a hydrogen atom in the above each formula according to a conventionally well-known method. For example, methods including 1) esterification by condensation of an alcohol with a fluorocarboxylic acid in the presence of an acid, 2) esterification by condensation of an alcohol with a fluorocarboxylic halide in the presence of a base, or the like are exemplified.

The structural unit (c1-3) is exemplified by a structural unit represented by the following formula (c1-3-1), and the like.

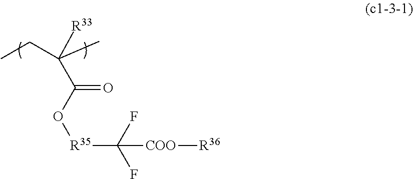

In the above formula (c1-3-1), R³³, R³⁵ and R³⁶ are as defined as in connection with the above formula (c1-3).

The compound that gives the structural unit represented by the above formula (c1-3-1) is exemplified by compounds represented by the following formulae, and the like.

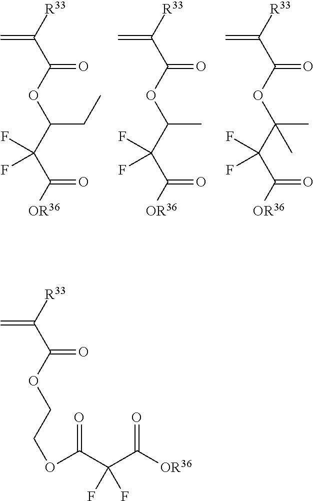

In the above formulae, R³³ and R³⁶ are as defined in connection with the above formula (c1-3).

A compound having the acid-dissociable group and/or base-dissociable group represented by R³⁶ in the above formulae can be synthesized, for example, using as a raw material a compound having a hydrogen atom represented by R³⁶ in the above each formula or a derivative thereof. By way of example, a compound having a group represented by the above formulae (19-2) to (19-4) as R³⁶ can be obtained, for example, by reacting a compound represented by the following formula (20) with a compound represented by the following formulae (21-1) to (21-3).

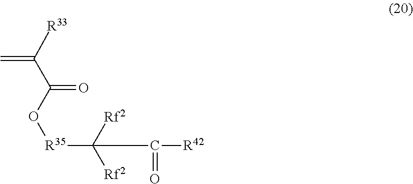

In the above formula (20), R³³, R³⁵ and Rf² are as defined in connection with the above formula (c1-3); and R⁴² represents a hydroxyl group or a halogen atom.

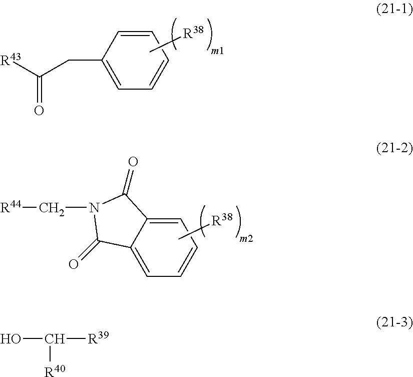

In the above formula (21-1) to formula (21-3), R³⁸, R³⁹, R⁴⁰, m1 and m2 are as defined in connection with the formula (19-1) to formula (19-3). In the formula (21-1), R⁴³ represents a halogen atom, and R⁴³ preferably represents Cl. In the formula (21-2), R⁴⁴ represents a halogen atom, and R⁴⁴ preferably represents Br.

Also, the intended compound can be obtained by reacting a compound represented by the following formula (22) with a compound represented by the following formula (23).

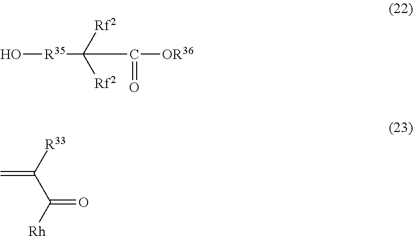

In the above formula (22), R³⁵, R³⁶ and Rf² are as defined in connection with the above formula (c1-3). In the above formula (23), R³³ is as defined in connection with the above formula (c1-3); and Rh represents a hydroxyl group or a halogen atom.

In the fluorine atom-containing polymer (C), the structural units (c1-1) to (c1-3) may be used either alone or two or more types in combination thereof. Among the structural units (c1-1) to (c1-3), at least two types are preferably contained and more preferably the structural unit (c1-2) and the structural unit (c1-3) are contained.

The fluorine atom-containing polymer (C) preferably further comprises a structural unit (hereinafter, may be also referred to as “structural unit (c2)”) having an acid-dissociable group other than the structural unit (c1), a structural unit (hereinafter, may be also referred to as “structural unit (c3)”) having an alkali-soluble group and excluding the structural unit (c1), or a structural unit (hereinafter, may be also referred to as “structural unit (c4)”) having an alkali-reactive group and excluding the structural unit (c1). Hereinafter, each structural unit will be explained in detail.

[Structural Unit (c2)]

The structural unit (c2) is exemplified by a structural unit represented by the following formula (24), and the like.

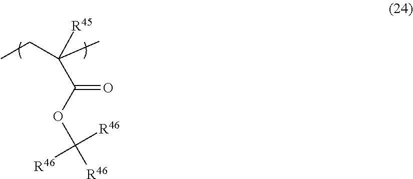

In the above formula (24), R⁴⁵ represents a hydrogen atom, a methyl group, a trifluoromethyl group or a hydroxymethyl group; R⁴⁶ each independently represents a linear or branched alkyl group having 1 to 4 carbon atoms, or an alicyclic hydrocarbon group having 4 to 20 carbon atoms or a derived group therefrom, wherein any two of three R⁴⁶s may taken together represent a bivalent alicyclic hydrocarbon group having 4 to 20 carbon atoms together with the carbon atom to which the R⁴⁶s bond or a derived group therefrom and remaining R⁴⁶ may be a linear or branched alkyl group having 1 to 4 carbon atoms, an alicyclic hydrocarbon group or having 4 to 20 carbon atoms or a derived group therefrom.

In the above formula (24), the alkyl group having 1 to 4 carbon atoms and the monovalent alicyclic hydrocarbon group having 4 to 20 carbon atoms represented by R⁴⁶, and the bivalent alicyclic hydrocarbon group having 4 to 20 carbon atoms represented by any two of three R⁴⁶s taken together or a derived group therefrom may be similarly defined to those for the R²¹ in the above formula (14).

The structural unit (c2) is preferably a structural unit represented by the following formula (24-1).

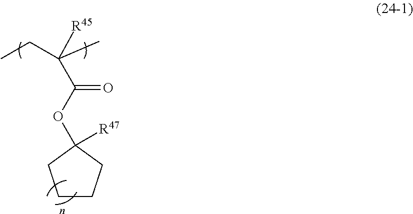

In the above formula (24-1), R⁴⁵ is as defined in connection with the formula (24); R⁴⁷ represents a linear or branched alkyl group having 1 to 4 carbon atoms; and n is an integer of 1 to 4.

Examples of the alkyl group having 1 to 4 carbon atoms represented by the R⁴⁷ include a methyl group, an ethyl group, an n-propyl group, an isopropyl group, an n-butyl group, a 2-methylpropyl group, a 1-methylpropyl group, a tert-butyl group, and the like.

The structural unit (c2) may be used either alone or two or more types in combination thereof. When the fluorine atom-containing polymer (C) includes the structural unit (c2), the difference between an advancing contact angle and a receding contact angle on a photoresist film can be decreased, thereby capable of leading to a response to an increase in scanning speed during liquid immersion lithography.

A monomer that gives the structural unit (c2) may be similar to the monomer that gives the structural unit (b1). The monomer that gives the structural unit (c2) is preferably a (meth)acrylic acid 2-methyl-2-cyclopentyl ester, a (meth)acrylic acid 2-ethyl-2-cyclopentyl ester, a (meth)acrylic acid 2-isopropyl-2-cyclopentyl ester, a (meth)acrylic acid 2-methyl-2-cyclohexyl ester, a (meth)acrylic acid 2-ethyl-2-cyclohexyl ester, or a (meth)acrylic acid 2-ethyl-2-cyclooctyl ester.

[Structural Unit (c3)]

An alkali-soluble group included in (c3) a structural unit is preferably a functional group having a pKa of 4 to 11 and a hydrogen atom in light of enhancement of solubility in a developing solution. The functional group is exemplified by groups represented by the following formulae (25) and (26).

—NHSO₂R⁴⁸ (25)

—COOH (26)

In the above formula (25), R⁴⁸ is a hydrocarbon group having 1 to 10 carbon atoms in which at least one hydrogen atom is substituted by fluorine atom(s). R⁴⁸ preferably represents a trifluoromethyl group.

The main chain moiety of the structural unit (c3) preferably include a structure derived from a (meth)acryloyl group, or an α-trifluoromethacryloyl group. In addition, the alkali-soluble group is preferably bonded directly or indirectly to —COO or the like in the main chain moiety.

The structural unit (c3) is exemplified by structural units represented by the following formulae (25-1) and (26-1), and the like.

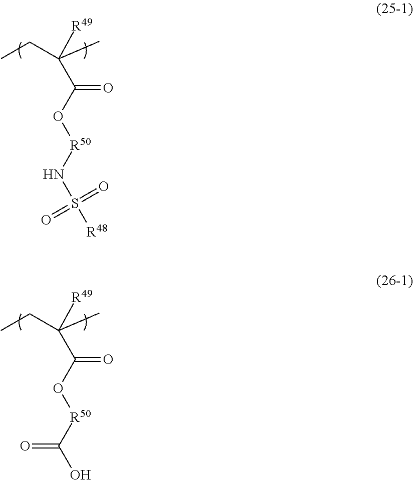

In the above formulae (25-1) and (26-1), R⁴⁹ represents a hydrogen atom, a methyl group or a trifluoromethyl group; R⁵⁰ represents a single bond, or a linear, branched or cyclic bivalent saturated hydrocarbon group or unsaturated hydrocarbon group having 1 to 20 carbon atoms; and R⁴⁸ represents a hydrocarbon group having 1 to 10 carbon atoms in which at least one hydrogen atom is substituted by a fluorine atom.

Examples of the linear and branched bivalent saturated hydrocarbon group and unsaturated hydrocarbon group represented by the R⁵⁰ having 1 to 20 carbon atoms include hydrocarbon groups derived from linear and branched alkyl groups and alkenyl groups having 1 to 20 carbon atoms such as a methyl group, an ethyl group, an n-propyl group, an isopropyl group, an n-butyl group, a 2-methylpropyl group, a 1-methylpropyl group, a tert-butyl group, a pentyl group, an isopentyl group, a neopentyl group, a hexyl group, a heptyl group, an octyl group, a nonyl group and a decyl group, and the like.

The bivalent cyclic saturated hydrocarbon group and unsaturated hydrocarbon group represented by the R⁵⁰ is exemplified by a group derived from an alicyclic hydrocarbon and an aromatic hydrocarbon having 3 to 20 carbon atoms, and the like. Examples of the alicyclic hydrocarbon having 3 to 20 carbon atoms include cycloalkanes such as cyclobutane, cyclopentane, cyclohexane, bicyclo[2.2.1]heptane, bicyclo[2.2.2]octane, tricyclo[5.2.1.0^{2,6}]decane, tricyclo[3.3.1.1^{3.7}]decane, tetracyclo[6.2.1.1^{3,6}.0^{2,7}]dodecane, and the like. Examples of the aromatic hydrocarbon include benzene, naphthalene, and the like.

It is to be noted that in the case in which R⁵⁰ represents a saturated hydrocarbon group or an unsaturated hydrocarbon group, the R⁵⁰ may be a group in which at least one hydrogen atom is substituted by a linear, branched or cyclic alkyl group having 1 to 12 carbon atoms such as a methyl group, an ethyl group, an n-propyl group, an isopropyl group, an n-butyl group, a 2-methylpropyl group, a 1-methylpropyl group or a tert-butyl group, a hydroxyl group, a cyano group, a hydroxyalkyl group having 1 to 10 carbon atoms, a carboxyl group, an oxygen atom, or the like. The R⁴⁸ in the above formula (25-1) may be similarly defined to that in connection with the above formula (25).

The structural unit (c3) may be used either alone or two or more types in combination thereof. When the fluorine atom-containing polymer (C) includes the structural unit (c3), solubility in a developing solution can be enhanced.

[Structural Unit (c4)]

The structural unit (c4) is exemplified by a structural unit having a lactone skeleton and a structural unit having a cyclic carbonate skeleton, and the like.

The structural unit having a lactone skeleton is exemplified by structural units represented by the above formulae (17-1) to (17-6), and the like. The structural unit having a cyclic carbonate skeleton is exemplified by a structural unit represented by the following formula (28), and the like.

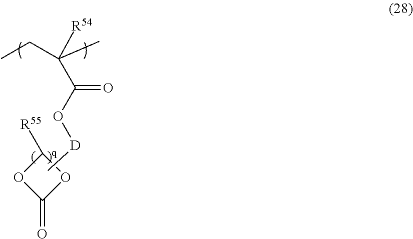

In the above formula (28), R⁵⁴ represents a hydrogen atom, a methyl group or a trifluoromethyl group; R⁵⁵ each independently represents a hydrogen atom or a chain hydrocarbon group having 1 to 5 carbon atoms; D represents a single bond, a bivalent or trivalent chain hydrocarbon group having 1 to 30 carbon atoms, a bivalent or trivalent alicyclic hydrocarbon group having 3 to 30 carbon atoms, or a bivalent or trivalent aromatic hydrocarbon group having 6 to 30 carbon atoms, wherein provided that D represents a trivalent group, a carbon atom included in D and a carbon atom that constitutes a cyclic carbonic acid ester may taken together represent a ring structure; and q is an integer of 2 to 4.

The cyclic carbonate structure corresponds to a 5-membered ring structure when q is 2 (ethylene group), a 6-membered ring structure when q is 3 (propylene group), and a 7-membered ring structure when q is 4 (butylene group).

Examples of the chain hydrocarbon group having 1 to 5 carbon atoms represented by the R⁵⁵ include a methyl group, an ethyl group, a propyl group, an isopropyl group, an n-butyl group, an isobutyl group, a sec-butyl group, a tert-butyl group, an n-pentyl group, a neopentyl group, and the like.

In the above formula (28), when D represents a single bond, an oxygen atom derived from a (meth)acrylic acid moiety that constitutes a main chain of the polymer and a carbon atom that forms the cyclic carbonate structure are bonded directly.

In the above formula (28), the chain hydrocarbon group represented by D is referred to a hydrocarbon group constituted with only a chain structure without including a cyclic structure. In addition, the alicyclic hydrocarbon group is referred to a hydrocarbon group that includes only an alicyclic hydrocarbon structure without including an aromatic ring structure in a ring structure thereof. However, the alicyclic hydrocarbon group is not necessarily constituted with only an alicyclic hydrocarbon structure and may include a chain structure in a part thereof. Furthermore, the aromatic hydrocarbon group as referred to means a hydrocarbon group that includes an aromatic ring structure in its ring structure. However, the aromatic hydrocarbon group is not necessarily constituted with only an aromatic ring structure and may include a chain structure or an alicyclic hydrocarbon structure in a part thereof.

The bivalent chain hydrocarbon group represented by D having 1 to 30 carbon atoms may be similarly defined in connection with R³¹. In addition, the trivalent chain hydrocarbon group having 1 to 30 carbon atoms is exemplified by a group obtained by eliminating one hydrogen atom from the group defined in connection with R³¹, and the like.

The bivalent alicyclic hydrocarbon group represented by D may be similarly defined in connection with R³¹. In addition, the trivalent alicyclic hydrocarbon group is exemplified by a group obtained by eliminating one hydrogen atom from the group defined in connection with R³¹, and the like.

Also, the bivalent aromatic hydrocarbon group represented by D may be similarly defined in connection with the R³¹. In addition, the trivalent aromatic hydrocarbon group is exemplified by a group obtained by eliminating one hydrogen atom from the group defined in connection with R³¹, and the like.

The structural unit having a cyclic carbonate skeleton is exemplified by a structural unit represented by the following formulae (28-1) to (28-22), and the like.

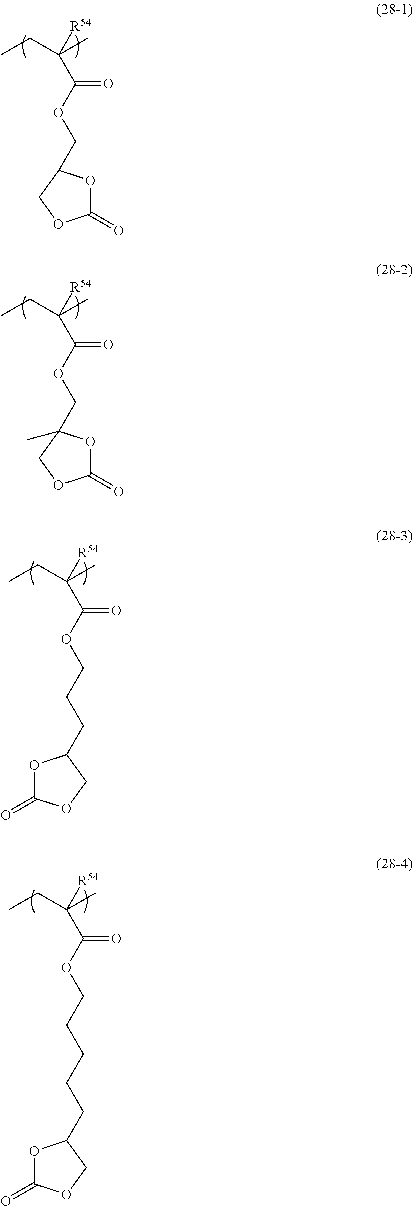

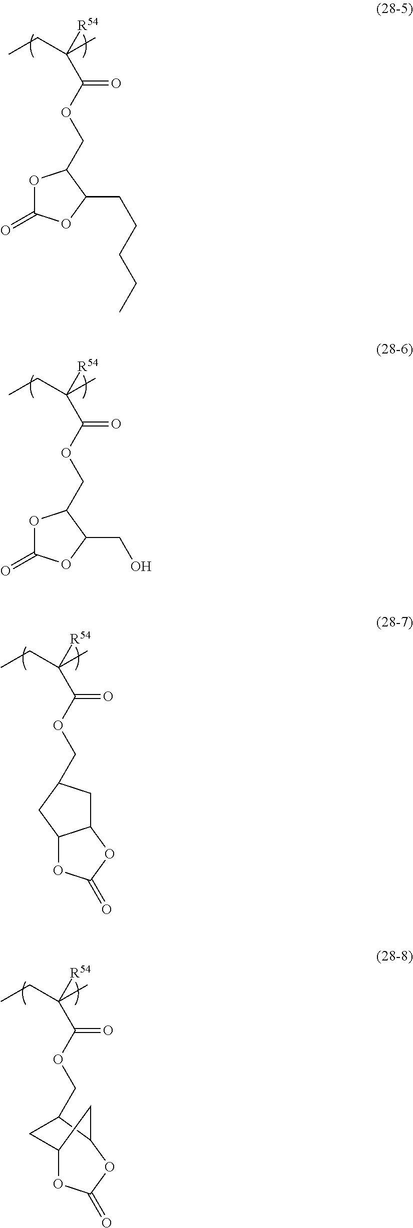

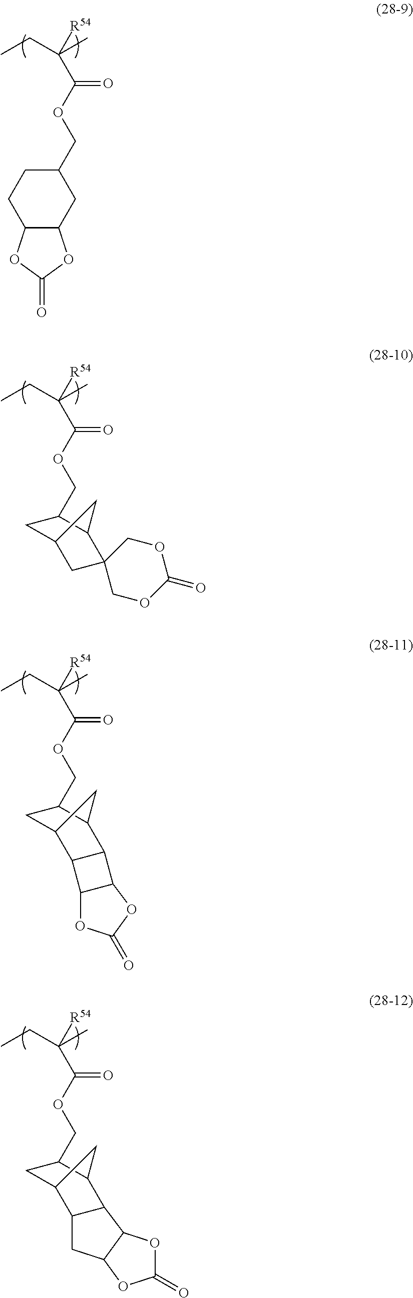

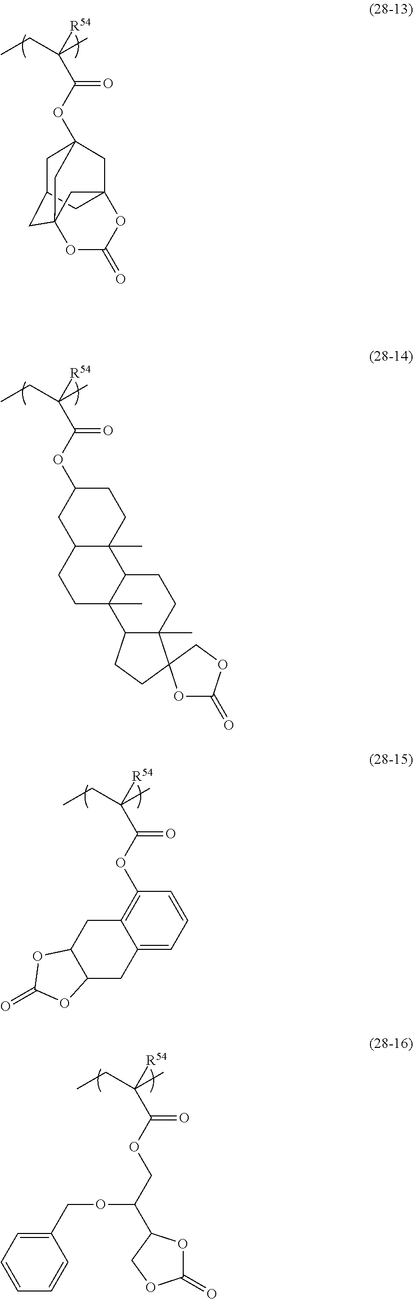

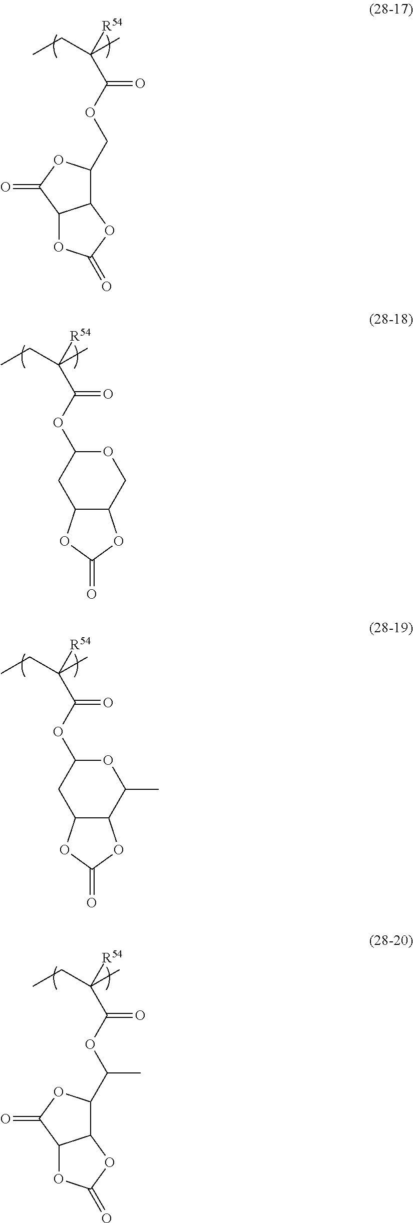

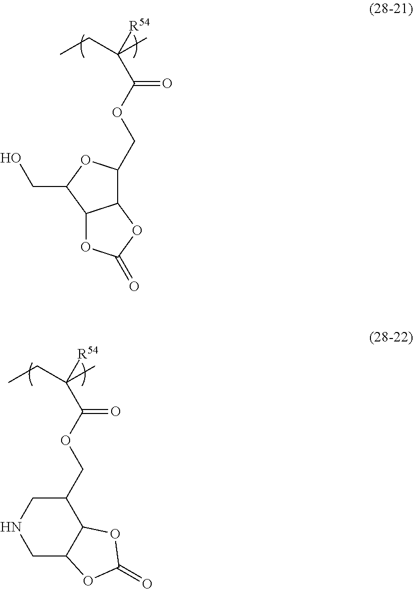

In the above formula, R⁵⁴ is as defined in connection with the formula (28).

The monomer that gives the structural unit represented by the above formula (28) can be synthesized by well-known methods, for example, methods described in Tetrahedron Letters, Vol. 27, No. 32, p. 3741 (1986), Organic Letters, Vol. 4, No. 15, p. 2561 (2002), etc.

The content of the structural unit (c1) is preferably 20 mol % to 90 mol %, more preferably 20 mol % to 80 mol % and particularly preferably 20 mol % to 70 mol % with respect to the total of the structural units. When the content of the structural unit (c1) falls within the above specific range, elution of the acid generating agent and an acid diffusion control agent, and the like in the photoresist film into a liquid for immersion lithography can be inhibited. In addition, due to an increase in a receding contact angle between the photoresist film and the liquid for immersion lithography, water droplets derived from the liquid for immersion lithography are less likely to remain on the photoresist film, thereby enabling defects caused by the liquid for immersion lithography to be efficiently inhibited.

The content of the structural unit (c2) is preferably no greater than 80 mol %, more preferably 20 mol % to 80 mol % and particularly preferably 30 mol % to 70 mol % with respect to the total of the structural units. The content of the structural unit (c2) falling within the above specific range is favorable in that the difference between an advancing contact angle and a receding contact angle can be decreased, and thus followability of a liquid immersion liquid is improved in liquid immersion lithography, thereby enabling a response to high-speed scanning.

The content of the structural unit (c3) is preferably no greater than 50 mol %, more preferably 5 mol % to 30 mol % and particularly preferably 5 mol % to 20 mol % with respect to the total of the structural units. The content of the structural unit (c3) falling within the above specific range enables both ensuring of water repellency after application and enhancement of an affinity to a developing solution during development.

The content of the structural unit (c4) is preferably no greater than 50 mol %, more preferably 5 mol % to 30 mol % and particularly preferably 5 mol % to 20 mol % with respect to the total of the structural units. The content of the structural unit (c4) falling within the above specific range enables both ensuring of water repellency after application and enhancement of an affinity to a developing solution during development.

### <Synthesis Method of (C) Fluorine Atom-Containing Polymer>

As a method for synthesizing the fluorine atom-containing polymer (C), for example, a method of producing the acid-dissociable group-containing polymer (B1) can be suitably applied.

The Mw of the fluorine atom-containing polymer (C) is preferably 1,000 to 50,000, more preferably 1,000 to 40,000 and particularly preferably 1,000 to 30,000 in terms of the polystyrene equivalent according to a GPC method. When the Mw is less than 1,000, a photoresist film having a sufficient receding contact angle may not be obtained. To the contrary, when the Mw exceeds 50,000, developability of the photoresist film may be decreased. The Mw/Mn is preferably 1 to 5 and more preferably 1 to 4.

The content of a fluorine atom in the fluorine atom-containing polymer (C) is not particularly limited as long as the content of a fluorine atom in the fluorine atom-containing polymer (C) is greater than that of the polymer (B). The content of a fluorine atom in the fluorine atom-containing polymer (C) is typically no less than 5% by mass, preferably 5% by mass to 50% by mass and more preferably 5% by mass to 40% by mass with respect to 100% by mass of the total of the fluorine atom-containing polymer (C).

### <Other Optional Components>

The composition may include in addition to the acid generating agent (A), the polymer (B) and the fluorine atom-containing polymer (C) described above, other optional components such as other acid generating agents, an acid diffusion control agent, a surfactant, a lactone compound, a crosslinking agent and/or an alicyclic additive as needed within the range not to impair the effects of the present invention. It is to be noted that the other optional components may be used either in combination of each component, or two or more types of each component may be contained. Hereinafter, other optional components will be described in detail.

### [Other Acid Generating Agent]

The other acid generating agent is exemplified by a radiation-sensitive acid generating agent other than the acid generating agent (A) and may include, for example, a compound described in Japanese Unexamined Patent Application, Publication No. 2009-134088, and the like.

The other acid generating agents are preferably triphenylsulfoniumtrifluoromethanesulfonate, triphenylsulfoniumnonafluoro-n-butanesulfonate, triphenylsulfoniumperfluoro-n-octanesulfonate, cyclohexyl 2-oxocyclohexylmethylsulfoniumtrifluoromethanesulfonatenesulfonate, dicyclohexyl 2-oxocyclohexylsulfoniumtrifluoromethanesulfonate, 2-oxocyclohexyldimethylsulfoniumtrifluoromethanesulfonate, 4-hydroxy-1-naphthyldimethylsulfoniumtrifluoromethanesulfonate, 4-hydroxy-1-naphthyltetrahydrothiopheniumtrifluoromethanesulfonate, 4-hydroxy-1-naphthyltetrahydrothiopheniumnonafluoro-n-butanesulfonate, 4-hydroxy-1-naphthyltetrahydrothiopheniumperfluoro-n-octanesulfonate, 1-(1-naphthylacetomethyl)tetrahydrothiopheniumtrifluoromethanesulfonate, 1-(1-naphthylacetomethyl)tetrahydrothiopheniumnonafluoro-n-butanesulfonate, 1-(1-naphthylacetomethyl)tetrahydrothiopheniumperfluoro-n-octanesulfonate, 1-(3,5-dimethyl-4-hydroxyphenyl)tetrahydrothiopheniumtrifluoromethanesulfonate, 1-(3,5-dimethyl-4-hydroxyphenyl)tetrahydrothiopheniumnonafluoro-n-butanesulfonate, and 1-(3,5-dimethyl-4-hydroxyphenyl)tetrahydrothiopheniumperfluoro-n-octanesulfonate.

The content of the other acid generating agent, in terms of the total content of the acid generating agent (A) and the other acid generating agent, is preferably 0.1 parts by mass to 30 parts by mass, more preferably 2 parts by mass to 27 parts by mass and particularly preferably 5 parts by mass to 25 parts by mass with respect to 100 parts by mass of the polymer (B). When the content is less than 0.1 parts by mass, sensitivity or resolution as a photoresist film may be decreased. To the contrary, when the content exceeds 30 parts by mass, coating properties as a photoresist film or pattern configuration may be decreased.

### [Acid Diffusion Control Agent]

An acid diffusion control agent is exemplified by a compound represented by the following formula (29) (hereinafter, may be also referred to as “nitrogen-containing compound (I)”), a compound having two nitrogen atoms in one molecule (hereinafter, may be also referred to as “nitrogen-containing compound (II)”), a compound having three or more nitrogen atoms (hereinafter, may be also referred to as “nitrogen-containing compound (III)”), an amide group-containing compound, a urea compound, a nitrogen-containing heterocyclic compound, and the like.

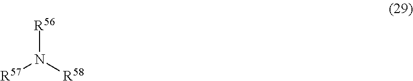

In the above formula (29), R⁵⁶ to R⁵⁸ each independently represent a hydrogen atom, an linear, branched or cyclic alkyl group, aryl group, aralkyl group or acid-dissociable group which are optionally substituted. Of these acid diffusion control agents, the nitrogen-containing compound (I), the nitrogen-containing compound (II) and the nitrogen-containing heterocyclic compound are preferred. Due to including the acid diffusion control agent, resist pattern configuration and dimension fidelity can be improved.

Of the nitrogen-containing compound (I), a nitrogen-containing compound not having any acid-dissociable groups is exemplified by trialkylamines such as tri-n-hexylamine, tri-n-heptylamine and tri-n-octylamine, and the like. Of the nitrogen-containing compound (I), the nitrogen-containing compound having an acid-dissociable group is exemplified by N-t-butoxycarbonyl-4-hydroxypiperidine, N-t-butoxycarbonylpyrrolidine, N-t-butoxycarbonyl-N′,N″-dicyclohexylamine, and the like.

The nitrogen-containing compound (II) is exemplified by N,N,N′,N′-tetrakis (2-hydroxypropyl)ethylene diamine, and the like. The nitrogen-containing compound (III) is exemplified by polymers such as polyethyleneimine, polyallylamine and dimethylaminoethylacrylamide, and the like. The nitrogen-containing heterocyclic compound is exemplified by 2-phenylbenzimidazole, N-t-butoxycarbonyl-2-phenylbenzimidazole, and the like.

In addition, as the acid diffusion control agent, a compound represented by the following formula (D1-0) may be also used.

X⁺Z⁻ (D1-0)

In the above formula (D1-0), X⁺ is a cation represented by the following formula (D1-1) or (D1-2); Z⁻ represents OH⁻, R^{D1}—COO⁻, or R^{D1}—SO₃⁻; and R^{D1} represents an optionally substituted alkyl group, alicyclic hydrocarbon group or aryl group.

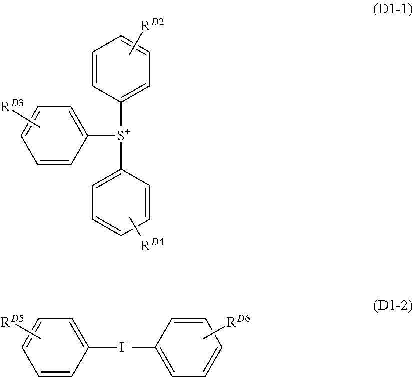

In the above formula (D1-1), R^{D2} to R^{D4} each independently represent a hydrogen atom, an alkyl group, an alkoxyl group, a hydroxyl group, or a halogen atom. In the above formula (1-2), R^{D5} and R^{D6} each independently represent a hydrogen atom, an alkyl group, an alkoxyl group, a hydroxyl group, or a halogen atom.

The compound is used as an acid diffusion control agent that is degraded by exposure and lose acid diffusion controllability (hereinafter, may be also referred to as “photodegradable acid diffusion control agent”). Due to including the compound, an acid is diffused at sites exposed with light whereas diffusion of an acid is controlled at sites not exposed with light, thereby resulting in excellent contrast between the site exposed with light and the site not exposed with light (i.e., achievement of clear boundary between the sites exposed and not exposed with light); therefore, in particular, LWR and MEEF of the composition can be effectively improved.

The R^{D2} to R^{D4} preferably represent a hydrogen atom, an alkyl group, an alkoxy group or a halogen atom since the R^{D2} to R^{D4} have an effect to decrease solubility of the compound into a developing solution. The R^{D5} and R^{D6} preferably represent a hydrogen atom, an alkyl group or a halogen atom.

Examples of the optionally substituted alkyl group represented by the R^{D1} include groups having one or more types of substituents such as hydroxyalkyl groups having 1 to 4 carbon atoms such as a hydroxymethyl group, a 1-hydroxyethyl group, a 2-hydroxyethyl group, a 1-hydroxypropyl group, a 2-hydroxypropyl group, a 3-hydroxypropyl group, a 1-hydroxybutyl group, a 2-hydroxybutyl group, a 3-hydroxybutyl group and a 4-hydroxybutyl group; alkoxyl groups having 1 to 4 carbon atoms such as a methoxy group, an ethoxy group, an n-propoxy group, an i-propoxy group, an n-butoxy group, a 2-methylpropoxy group, a 1-methylpropoxy group and a t-butoxy group; a cyano group; cyanoalkyl groups having 2 to 5 carbon atoms such as a cyanomethyl group, a 2-cyanoethyl group, a 3-cyanopropyl group and a 4-cyanobutyl group, and the like. Of these, a hydroxymethyl group, a cyano group and a cyanomethyl group are preferable.

The optionally substituted alicyclic hydrocarbon group represented by the R^{D1} is exemplified by a monovalent group derived from an alicyclic hydrocarbon having a cycloalkane skeleton such as hydroxycyclopentane, hydroxycyclohexane or cyclohexanone; a bridged alicyclic skeleton such as 1,7,7-trimethylbicyclo[2.2.1]heptan-2-one(camphor), or the like. Of these, a group derived from 1,7,7-trimethylbicyclo[2.2.1]heptan-2-one is preferable.

Examples of the optionally substituted aryl group represented by the R^{D1} include a phenyl group, a benzyl group, a phenylethyl group, a phenylpropyl group, a phenylcyclohexyl group, and the like. Of these, a phenyl group, a benzyl group and a phenylcyclohexyl group are preferable.

The R^{D1} is preferably an alicyclic hydrocarbon group or an aryl group since they have an effect of decreasing the solubility of the compound in a developing solution.

The Z⁻ is preferably an anion represented by the following formula (1a) or an anion represented by the following formula (1b).

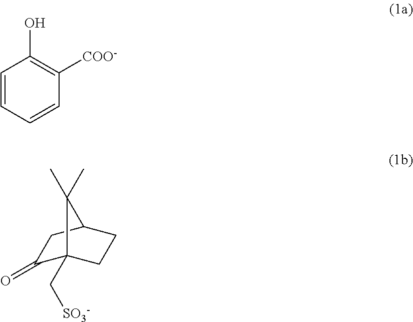

The photodegradable acid diffusion control agent is represented by the above formula (D1-0) and specifically a sulfonium salt compound or an iodonium salt compound that satisfies the requirements described above.

Examples of the sulfonium salt compound include triphenylsulfonium hydroxide, triphenylsulfonium acetate, triphenylsulfonium salicylate, diphenyl-4-hydroxyphenylsulfonium hydroxide, diphenyl-4-hydroxyphenylsulfonium acetate, diphenyl-4-hydroxyphenylsulfonium salicylate, triphenylsulfonium 10-camphorsulfonate, 4-t-butoxyphenyldiphenylsulfonium 10-camphorsulfonate, and the like.

Examples of the iodonium salt compound include bis(4-t-butylphenyl)iodonium hydroxide, bis(4-t-butylphenyl)iodonium acetate, bis(4-t-butylphenyl)iodonium hydroxide, bis(4-t-butylphenyl)iodonium acetate, bis(4-t-butylphenyl)iodonium salicylate, 4-t-butylphenyl-4-hydroxyphenyliodonium hydroxide, 4-t-butylphenyl-4-hydroxyphenyliodonium acetate, 4-t-butylphenyl-4-hydroxyphenyliodonium salicylate, bis(4-t-butylphenyl)iodonium 10-camphorsulfonate, diphenyliodonium 10-camphorsulfonate, and the like.

The content of the acid diffusion control agent is preferably no greater than 30 parts by mass and more preferably no greater than 20 parts by mass with respect to 100 parts by mass of the polymer (B). When the content of the acid diffusion control agent exceeds 30 parts by mass, sensitivity of a photoresist film formed tends to be significantly decreased.

### [Surfactant]

A surfactant is a component that exhibits an effect of improving a coating property, developability, and the like. Examples of the surfactant include nonionic surfactants such as polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate and polyethylene glycol distearate, and the like. Examples of commercially available products include KP341 (manufactured by Shin-Etsu Chemical Co., Ltd.), Polyflow No. 75 and Polyflow No. 95 (both manufactured by Kyoeisha Chemical Co., Ltd.), EFTOP EF301, EFTOP EF 303 and EFTOP EF 352 (all manufactured by Tochem Products Corporation), Megaface F171 and Megaface F173 (both manufactured by Dainippon Ink And Chemicals, Incorporated), Fluorad FC430 and Fluorad FC431 (both manufactured by Sumitomo 3M Limited), ASAHI GUARD AG710, Surflon S-382, Surflon SC-101, Surflon SC-102, Surflon SC-103, Surflon SC-104, Surflon SC-105 and Surflon SC-106 (all manufactured by Asahi Glass Co., Ltd.), and the like. The content of the surfactant is typically no greater than 2 parts by mass with respect to 100 parts by mass of the polymer (B).

### [Lactone Compound]

A lactone compound has an effect of efficiently segregating the fluorine atom-containing polymer (C) on the surface of the resist film. Due to including a lactone compound, the amount of the fluorine atom-containing polymer (C) added can be smaller than ever before. Therefore, elution of a component from a resist film to a liquid immersion liquid can be inhibited without impairing basic characteristics as a resist such as LWR, development defects and pattern collapse resistance, and water repellency of the surface of a resist film that inhibits defects derived from liquid immersion such as watermark defects can be maintained as a result of no remaining of droplets even if liquid immersion lithography is carried out by high-speed scanning.

Examples of the lactone compound include gamma-butyrolactone, valerolactone, mevalonic lactone, norbornanelactone, and the like.

The content of the lactone compound is preferably 30 parts by mass to 200 parts by mass and more preferably 50 parts by mass to 150 parts by mass with respect to 100 parts by mass of the fluorine atom-containing polymer (C).

### [Crosslinking Agent]

In the case in which the composition is used as a negative radiation-sensitive resin composition, a crosslinking agent that can crosslink an alkali-soluble polymer in the presence of an acid may be blended. A crosslinking agent is exemplified by a compound having one or more types of functional groups having crosslinking reactivity with an alkali-soluble polymer (i.e., crosslinkable functional group).

Examples of the crosslinkable functional group include a glycidyl ether group, a glycidyl ester group, a glycidylamino group, a methoxymethyl group, an ethoxymethyl group, a benzyloxymethyl group, an acetoxymethyl group, a benzoyloxymethyl group, a formyl group, an acetyl group, a vinyl group, an isopropenyl group, a (dimethylamino)methyl group, a (diethylamino)methyl group, a (dimethylolamino)methyl group, a (diethylolamino)methyl group, a morpholinomethyl group, and the like.

The crosslinking agent is exemplified by a crosslinking agent described in WO2009/51088, and the like. The crosslinking agent is preferably a methoxymethyl group-containing compound and more preferably dimethoxymethylurea and tetramethoxymethylglycoluril.

The content of the crosslinking agent is preferably 5 parts by mass to 95 parts by mass, more preferably 15 parts by mass to 85 parts by mass and particularly preferably 20 parts by mass to 75 parts by mass with respect to 100 parts by mass of the alkali-soluble polymer (B2). When the content of the crosslinking agent is less than 5 parts by mass, a decrease in the percentage of residual film, as well as meandering, swelling, etc., of the pattern are likely to occur. To the contrary, when the content exceeds 95 parts by mass, the alkali developability is likely to be decreased.

### [Alicyclic Additive]

An alicyclic additive is a component that exhibits an effect of additionally improving dry etching resistance, pattern configuration, adhesiveness to a substrate, and the like. Examples of the alicyclic additive include adamantane derivatives such as t-butyl 1-adamantanecarboxylate, t-butoxycarbonylmethyl 1-adamantanecarboxylate, di-t-butyl 1,3-adamantanedicarboxylate, t-butyl 1-adamantaneacetate, t-butoxycarbonylmethyl 1-adamantaneacetate, di-t-butyl 1,3-adamantanediacetate; deoxycholic acid esters such as t-butyl deoxycholate, t-butoxycarbonylmethyl deoxycholate, 2-ethoxyethyl deoxycholate, 2-cyclohexyloxyethyl deoxycholate, 3-oxocyclohexyl deoxycholate, tetrahydropyranyl deoxycholate, a deoxycholic acid mevalonolactone ester; lithocholic acid esters such as t-butyl lithocholate, t-butoxycarbonylmethyl lithocholate, 2-ethoxyethyl lithocholate, 2-cyclohexyloxyethyl lithocholate, 3-oxocyclohexyl lithocholate, tetrahydropyranyl lithocholate, a lithocholic acid mevalonolactone ester, and the like.

The content of the alicyclic additive is typically no greater than 50 parts by mass and preferably no greater than 30 parts by mass with respect to 100 parts by mass of the polymer (B).

### <Preparation Method of Radiation-Sensitive Resin Composition>

The composition is usually prepared to form a composition solution upon use by dissolving in a solvent so as to give the total solid content of 1% by mass to 50% by mass and preferably 3% by mass to 25% by mass, followed by filtration through a filter having a pore size of, for example, about 5 nm.

Examples of the solvent which may be used for preparing the composition include linear or branched ketones; cyclic ketones; propylene glycol monoalkyl ether acetates; alkyl 2-hydroxypropionates; alkyl 3-alkoxypropionates, and the like.

These solvents may be used either alone or two or more types in combination thereof.

### <Formation Method of a Resist Pattern>

The method for forming a resist pattern of the embodiment of the present invention includes:

(1) a step of forming a photoresist film on a substrate using the radiation-sensitive resin composition;

(2) a step of exposing the formed photoresist film through an immersion liquid; and

(3) a step of developing the exposed photoresist film to form a resist pattern.

In the method for forming a resist pattern, the composition is used as a photoresist composition, so that a resist pattern having favorable MEEF and LWR can be formed while development defects in a developing process are inhibited.

In the step (1), a photoresist film is formed by coating a solution of the composition on a substrate such as, for example, a silicon wafer, or a wafer coated with aluminum by an appropriate coating means such as means of spin coating, cast coating or roll coating. Specifically, after a solution of the radiation-sensitive resin composition is coated such that the resulting resist film has a predetermined film thickness, prebaking (PB) is carried out to allow the solvent in the coating film to be volatilized, whereby a resist film is formed.

The thickness of the resist film is preferably 10 to 5,000 nm and more preferably 10 to 2,000 nm.

The conditions of heating in PB may vary depending on the blend composition of the radiation-sensitive resin composition, and may involve preferably about 30° C. to 200° C. and more preferably 50° C. to 150° C.

In the step (2), a liquid for immersion lithography is provided on the photoresist film formed in the step (1), and a radioactive ray is irradiated through the liquid for immersion lithography to execute liquid immersion lithography of the photoresist film.

As the liquid for immersion lithography, for example, pure water, long chain or cyclic aliphatic compound or the like may be used. The radioactive ray employed is appropriately selected from visible light rays, ultraviolet rays, far ultraviolet rays, X-rays, charged particle rays and the like in accordance with the type of the acid generating agent used. The radioactive ray is preferably a far ultraviolet ray typified by an ArF excimer laser (wavelength: 193 nm) or a KrF excimer laser (wavelength: 248 nm), and particularly preferably an ArF excimer laser (wavelength: 193 nm).

Conditions of the exposure such as exposure dose may be appropriately determined in accordance with the blend composition of the radiation-sensitive resin composition and the type of the additives. In the embodiment of present invention, a heat treatment (PEB: post exposure baking) is preferably carried out after the exposure. The PEB enables a dissociation reaction of the acid-dissociable group in the resin components to smoothly proceed. Conditions of heating of the PEB may be appropriately adjusted depending on the blend composition of the radiation-sensitive resin composition, and involve usually 30° C. to 200° C., and preferably 50° C. to 170° C.

In the embodiment of present invention, in order to maximize the potential capability of the radiation-sensitive resin composition, an organic or inorganic antireflection film may be also formed on the substrate employed, as disclosed in, for example, Japanese Examined Patent, Publication No. H6-12452 (Japanese Unexamined Patent Application, Publication No. S59-93448), and the like. Moreover, in order to prevent influences of basic impurities etc., included in the environment atmosphere, a protective film may be also provided on the photoresist film, as disclosed in, for example, Japanese Unexamined Patent Application, Publication No. H5-188598, and the like. Furthermore, in order to prevent effluence of the acid generating agent etc., from the photoresist film during the liquid immersion lithography, a protective film for liquid immersion may be provided on the photoresist film, as disclosed in, for example, Japanese Unexamined Patent Application, Publication No. 2005-352384, and the like. It is to be noted that these techniques may be used in combination.

It is to be noted that in the method for forming a resist pattern by the liquid immersion lithography, the resist pattern can be formed with only the photoresist film obtained using the composition, without providing the protective film (upper layer film) described above on the photoresist film. If a resist pattern is formed with the photoresist film that is free from the upper layer film, a step of forming a protective film (upper layer film) can be omitted, thereby capable of leading to expectation for improvement of throughput.

In the step (3), a predetermined resist pattern is formed by subjecting the exposed resist film to development. Examples of preferable developer solution used in the development step include aqueous alkali solutions prepared by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethylammonium hydroxide, pyrrole, piperidine, choline, 1,8-diazabicyclo-[5.4.0]-7-undecene or 1,5-diazabicyclo-[4.3.0]-5-nonene.

The concentration of the alkaline aqueous solution is preferably no greater than 10% by mass. In the case in which the concentration of the alkaline aqueous solution is greater than 10% by mass, sites unexposed with light may be also dissolved in the developing solution. In addition, an organic solvent may be also added to the developing solution consisting of the aforementioned alkaline aqueous solution. Examples of the organic solvent include ketones such as acetone, methyl ethyl ketone, methyl-i-butyl ketone, cyclopentanone, cyclohexanone, 3-methyl cyclopentanone and 2,6-dimethyl cyclohexanone; alcohols such as methyl alcohol, ethyl alcohol, n-propyl alcohol, i-propyl alcohol, n-butyl alcohol, t-butyl alcohol, cyclopentanol, cyclohexanol, 1,4-hexanediol and 1,4-hexanedimethylol; ethers such as tetrahydrofuran and dioxane; esters such as ethyl acetate, n-butyl acetate and i-amyl acetate; aromatic hydrocarbons such as toluene and xylene, as well as phenol, acetonyl acetone, dimethylformamide, and the like. These organic solvents may be used either alone, or two or more types thereof may be used in combination. These organic solvents may be used either alone, or two or more types thereof may be used in combination.

The amount of the organic solvent contained is preferably no greater than 100 parts by volume with respect to 100 parts by volume of the alkaline aqueous solution. In the case in which the amount of the organic solvent used is greater than 100 parts by volume, developability is lowered, and thus development residues at the site exposed with light may increase. Moreover, to the developing solution consisting of the alkaline aqueous solution may be added an appropriate amount of a surfactant and the like. It is to be noted that the development with a developing solution consisting of the alkaline aqueous solution is, in general, followed by washing with water and drying.

### EXAMPLES

Hereinafter, the present invention is explained in detail by way of Examples, but the present invention is not limited thereto.

### <Synthesis of (A) Acid Generator>

### Synthesis Example 1

As a precursor of the acid generating agent (A), a compound, sodium 1,1,2,2-tetrafluoro-4-(3-hydroxyadamantane-1-carbonyloxy)butane-1-sulfonate, represented by the following formula (30) was synthesized by the following method.

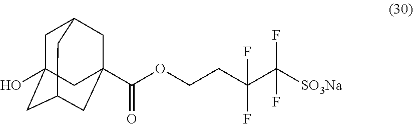

In a reaction flask, a mixture of 19.6 g of 3-hydroxyadamantane-1-carboxylic acid, 120 g of methanol, 100 g of dichloromethane, 100 g of ion exchanged water was stirred at 60° C. for 20 hrs. Further, to the reaction solution were added 8.1 g of chloromethoxymethane and 5 g of N,N-diisopropyl ethylamine, and the mixture was stirred at room temperature for 24 hrs. The temperature of the reaction solution was allowed to return to room temperature, and then a solution prepared by dissolving 10 g of sodium hydroxide into 90 g of ion exchanged water was added thereto, followed by stirring for 1 hour at room temperature. Thereafter, an organic layer was extracted and then washed with 500 g of ion exchanged water. The washed reaction liquid was concentrated in vacuo to give 27.9 g of a crude product of 3-methoxymethoxyadamantane-1-carboxylic acid. The reaction scheme is shown below.

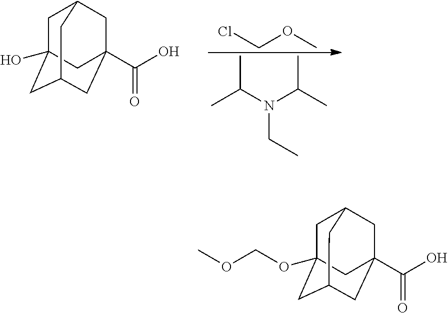

In a reaction flask, 1.0 g of tert-butoxypotassium was dissolved into 4.7 g of dimethyl sulfoxide, and 27.0 g of 3-methoxymethoxyadamantane-1-carboxylic acid and 100 g of dichloromethane were added and the mixture was stirred at 60° C. To the reaction solution, 28.7 g of 1,4-dibromo-1,1,2,2-tetrafluorobutane was added and the mixture was stirred for 4 hrs. After the reaction liquid was cooled to room temperature, 70 g of water was added and an organic layer was extracted, and the organic layer was washed with an aqueous solution prepared by dissolving 92.4 g of sodium bicarbonate into 500 mL of ion exchanged water three times and with 100 g of saturated saline twice. The organic layer was concentrated in vacuo to give 45.3 g of 4-bromo-3,3,4,4-tetrafluorobutyl 3-methoxymethoxyadamantane-1-carboxylate ester. The reaction scheme is shown below.

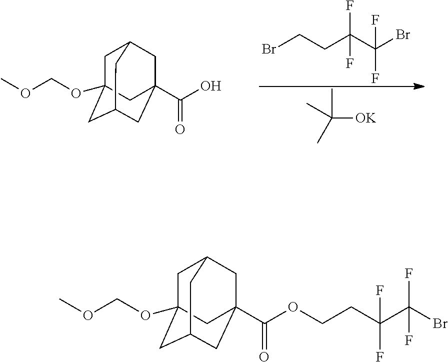

In a reaction flask, after 9.8 g of sodium dithionite and 7.1 g of sodium carbonate were charged, 50 mL of ion exchanged water was charged and the mixture was stirred for 30 min. Next, to the mixed solution 40.0 g of 4-bromo-3,3,4,4-tetrafluorobutyl 3-methoxymethoxyadamantane-1-carboxylate ester which had been dissolved into 100 g of dichloromethane was added dropwise over 5 min, and thereafter the mixture was heated at 60° C. for 3.5 hrs with stirring. The reaction solution was removed in vacuo to give 54.3 g of sodium 1,1,2,2-tetrafluoro-4-(3-methoxymethoxyadamantane-1-carbonyloxy)butane-1-sulfinate. The reaction scheme is shown below.

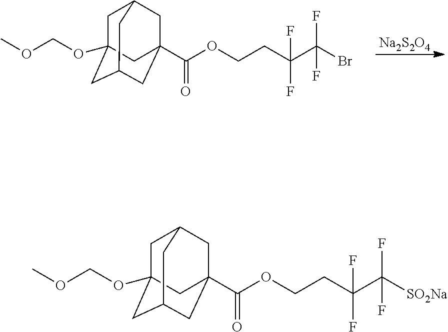

In a reaction flask, to sodium 1,1,2,2-tetrafluoro-4-(3-methoxymethoxyadamantane-1-carbonyloxy)butane-1-sulfinate were added ion exchanged water, 28.1 g of sodium carbonate and 0.92 g of sodium tungstate and the mixture was stirred for 30 min. Next, to the reaction mixture 30 mL of 30 wt % hydrogen peroxide water was added dropwise over 30 min, and then the mixture was stirred at 60° C. for 3 hrs. Next, a reaction solvent was removed in vacuo to give 87.9 g of a white solid of sodium 1,1,2,2-tetrafluoro-4-(3-methoxymethoxyadamantane-1-carbonyloxy)butane-1-sulfonate. The reaction scheme is shown below.

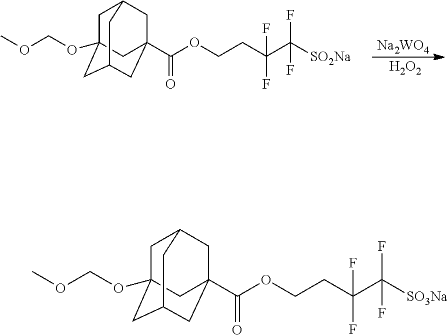

Into a reaction flask were charged 80.0 g of sodium 1,1,2,2-tetrafluoro-4-(3-methoxymethoxyadamantane-1-carbonyloxy)butane-1-sulfonate and 150 g of dichloromethane, and the mixture was stirred at 0° C. Then 50 g of 4 N sulfuric acid was added thereto dropwise at the same temperature over 20 min, and then the mixture was stirred at 0° C. for 1 hour. Next, an organic layer was extracted and washed with 100 g of ion exchanged which was thereafter removed in vacuo to give 35.0 g of sodium 1,1,2,2-tetrafluoro-4-(3-hydroxyadamantane-1-carbonyloxy)butane-1-sulfonate of interest. The reaction scheme is shown below.

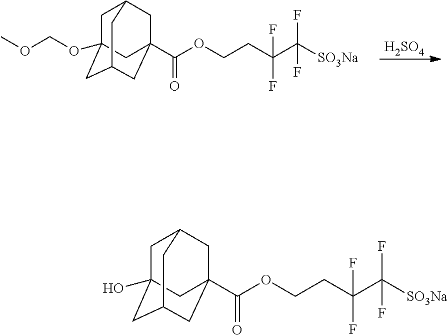

Note that an analysis was carried out with regard to sodium 1,1,2,2-tetrafluoro-4-(3-hydroxyadamantane-1-carbonyloxy)butane-1-sulfonate using ¹H-NMR (JNM-EX270, manufactured by JEOL, Ltd.). As a result, chemical shift obtained was as follows: ¹H-NMR [σppm (DMSO): 1.24 (2H,m), 1.36-1.47 (4H,m), 1.53-1.64 (4H,m), 1.84-2.03 (4H,m), 3.65 (1H,s), 4.08 (1H,m)], ¹⁹F-NMR [σppm (DMSO): 58.82 (m)]. Accordingly, the analysis confirmed that the sample was a compound of interest. For ¹H-NMR, the peak of sodium 3-trimethylsilylpropionate-2,2,3,3-d₄ was defined as 0 ppm (internal standard), and for ¹⁹F-NMR, the peak of hexafluorobenzene was defined as 0 ppm (internal standard). The purity was 93 wt % according to the ¹H-NMR. It is to be noted that instruments and conditions used for the ¹H-NMR, and conditions used for the ¹⁹F-NMR in the following Synthesis Examples were similar to the foregoings.

### Synthesis Example 2

A compound, triphenylsulfonium 4-(3-(2-tert-butoxy-2-oxoethoxy)adamantane-1-carbonyloxy)-1,1,2,2-tetrafluorobutane-1-sulfonate (hereinafter, may be also referred to as “(A-1)”) represented by the following formula (31) was synthesized by the following method.

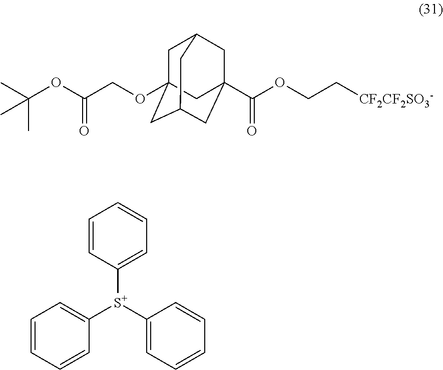

Into a reaction flask, 20.5 g of sodium 1,1,2,2-tetrafluoro-4-(3-hydroxyadamantane-1-carbonyloxy)butane-1-sulfonate obtained in Synthesis Example 1, 110.0 g of tertiary butyl chloroacetate, 100 g of dichloromethane and 20.0 g of a 1 N aqueous sodium hydroxide solution were charged, and the mixture was stirred at room temperature for 1 hour. An organic layer was extracted, followed by washing with 100 g of ion exchanged water five times, and then a solvent was removed in vacuo to give 20.3 g of sodium 4-(3-(2-tertiarybutoxy-2-oxoethoxy)adamantane-1-carbonyloxy)-1,1,2,2-tetrafluorobutane-1-sulfonate. The reaction scheme is shown below.

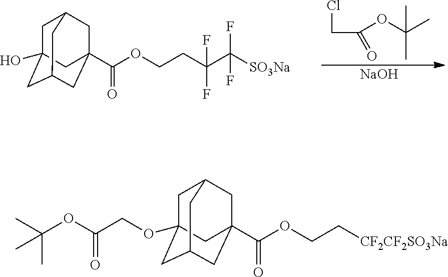

Into a reaction flask were charged 20.0 g of sodium 4-(3-(2-tertiarybutoxy-2-oxoethoxy)adamantane-1-carbonyloxy)-1,1,2,2-tetrafluorobutane-1-sulfonate and 15.0 g of triphenylsulfoniumbromide, 100 g of ion exchanged water and 100 g of dichloromethane, and the mixture was stirred at room temperature for 1 hour. An organic layer was extracted, and then was washed with 100 g of ion exchanged water five times. Thereafter, a solvent was eliminated to give 28.7 g of triphenylsulfonium4-(3-(2-tertiarybutoxy-2-oxoethoxy)adamantane-1-carbonyloxy)-1,1,2,2-tetrafluorobutane-1-sulfonate. The reaction scheme is shown below.

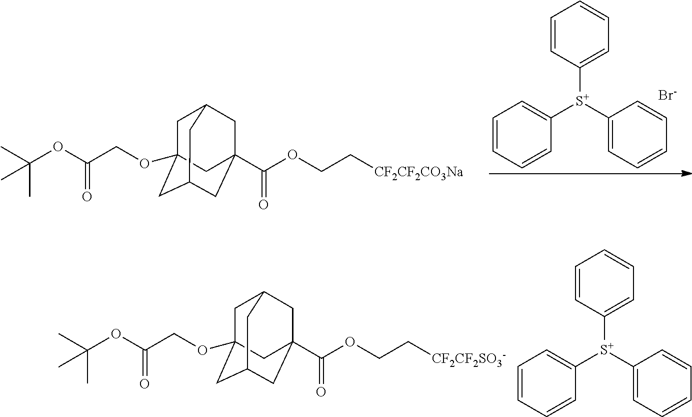

Note that an analysis was carried out with regard to triphenylsulfonium 4-(3-(2-tert-butoxy-2-oxoethoxy)adamantane-1-carbonyloxy)-1,1,2,2-tetrafluorobutane-1-sulfonate using the ¹H-NMR. As a result, chemical shift obtained was as follows: ¹H-NMR [σppm (DMSO): 1.24 (2H,m), 1.36-1.47 (7H,m), 1.53-1.64 (4H,m), 1.84-2.03 (4H,m), 4.08 (1H,m), 4.33 (1H, s), 7.76-7.89 (15H,m)], ¹⁹F-NMR [σppm (DMSO) : 58.82 (m)]. Accordingly, the analysis confirmed that the sample was a compound of interest. The purity was no less than 99 wt %.

### Synthesis Example 3

A compound, triphenylsulfonium 1,1,2,2-tetrafluoro-4-(3-(2,2,2-trifluoroacetoxy)adamantane-1-carbonyloxy)-butane-1-sulfonate (hereinafter, may be also referred to as “(A-2)”) represented by the following formula (32) was synthesized by the following method.

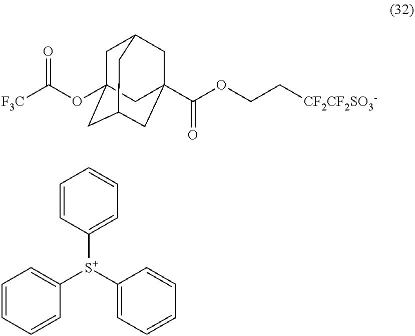

Into a reaction flask were added 20.5 g of sodium 1,1,2,2-tetrafluoro-4-(3-hydroxyadamantane-1-carbonyloxy)butane-1-sulfonate obtained in Synthesis Example 1 and 100 g of dichloromethane. 11.5 g of trifluoroacetic anhydride was added over 30 min with cooling to 0° C. on an ice bath while stirring. Thereafter, 5.3 g of triethylamine was added with enough stirring. Subsequently, an organic layer was extracted, followed by washing with saturated saline, and then a solvent was removed in vacuo to give 24.2 g of sodium 1,1,2,2-tetrafluoro-4-(3-(2,2,2-trifluoroacetoxy)adamantane-1-carbonyloxy)-butane-1-sulfonate. The reaction scheme is shown below.

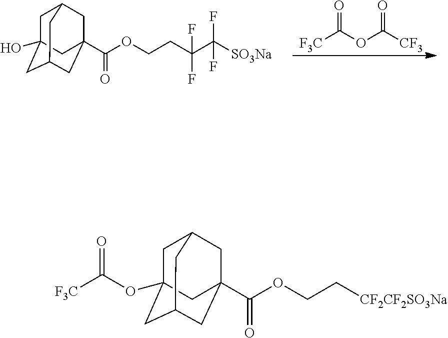

Into a reaction flask were charged 20.0 g of sodium 1,1,2,2-tetrafluoro-4-(3-(2,2,2-trifluoroacetoxy)adamantane-1-carbonyloxy)-butane-1-sulfonate and 15.0 g of triphenylsulfoniumbromide, 100 g of ion exchanged water and 100 g of dichloromethane, and the mixture was stirred at room temperature for 1 hour. After an organic layer was separated, the organic layer was washed with 100 g of ion exchanged water five times. Thereafter, a solvent was removed in vacuo to give 27.3 g of triphenylsulfonium 1,1,2,2-tetrafluoro-4-(3-(2,2,2-trifluoroacetoxy)adamantane-1-carbonyloxy)-butane-1-sulfonate. The reaction scheme is shown below.

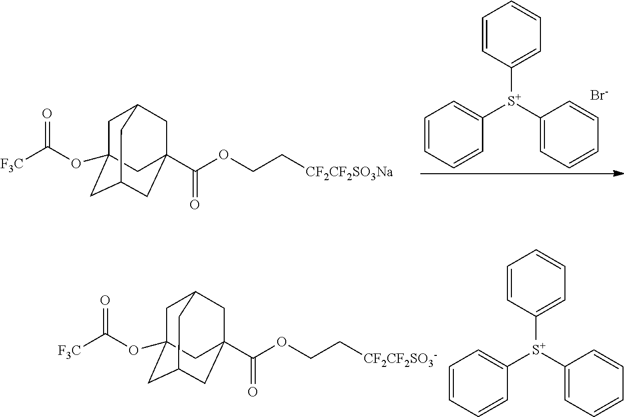

Note that an analysis was carried out with regard to triphenylsulfonium 1,1,2,2-tetrafluoro-4-(3-(2,2,2-trifluoroacetoxy)adamantane-1-carbonyloxy)-butane-1-sulfonate using ¹H-NMR. As a result, chemical shift obtained was as follows: ¹H-NMR [σppm (DMSO): 1.18 (3H,m), 1.38-1.40 (3H,m), 1.56 (3H,m), 1.76-1.89 (4H,m), 2.15 (1H,s), 4.08 (1H,m), 7.76-7.89 (15H,m)], ¹⁹F-NMR [σppm (DMSO):58.82 (m)]. Accordingly, the analysis confirmed that the sample was a compound of interest. The purity was no less than 99 wt %.

### Synthesis Example 4

A compound, triphenylsulfonium 4-(3-(2-ethoxy-1,1-difluoro-2-oxoethoxy)adamantane-1-carbonyloxy)-1,1,2,2-tetrafluorobutane-1-sulfonate (hereinafter, may be also to be referred to “(A-3)”) represented by the following formula (33) was synthesized by the following method.

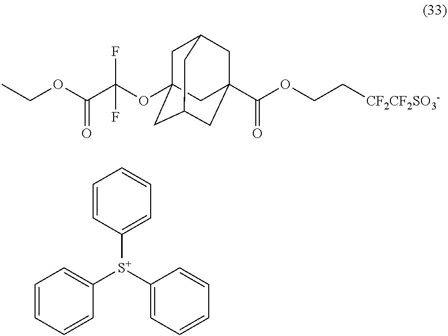

Into a reaction flask were added 20.5 g of sodium 1,1,2,2-tetrafluoro-4-(3-hydroxyadamantane-1-carbonyloxy)butane-1-sulfonate obtained in Synthesis Example 1, 100 g of dichloromethane and 20.0 g of 1 N aqueous potassium hydroxide solution. The temperature of the reaction flask was adjusted to 40° C. on a water bath and 6.5 g of chloro-2,2-difluoroacetic acid was added dropwise over 5 min with stirring. Then the reaction was allowed for 40 hrs. Thereafter, an organic layer was extracted, followed by washing with 100 g of ion exchanged water, and then a solvent was removed in vacuo to give 23.7 g of sodium 4-(3-(carboxydifluoromethoxy)adamantane-1-carbonyloxy)-1,1,2,2-tetrafluorobutane-1-sulfonate. The reaction scheme is shown below.

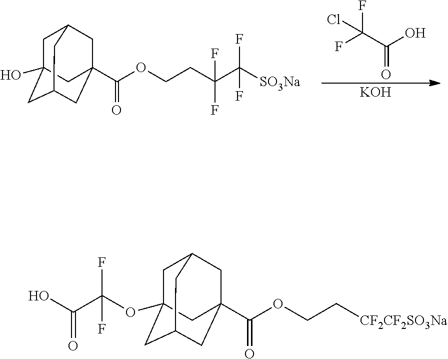

Into a reaction flask were added 23.0 g of sodium 4-(3-(carboxydifluoromethoxy)adamantane-1-carbonyloxy)-1,1,2,2-tetrafluorobutane-1-sulfonate, 100 g of dichloromethane, 10 g of ethanol and 50 g of 4 N sulfuric acid, and the mixture was stirred. The temperature of the reaction flask was adjusted to 40° C. on a water bath and the reaction was allowed for 1 hour. Thereafter, a solution obtained by dissolving 92.41 g of sodium bicarbonate into 500 g of water was added with enough stirring. Subsequently, an organic layer was extracted, followed by washing with 100 g of ion exchanged water three times, and then a solvent was removed in vacuo to give 21.3 g of sodium 4-(3-(2-ethoxy-1,1-difluoro-2-oxoethoxy)adamantane-1-carbonyloxy)-1,1,2,2-tetrafluorobutane-1-sulfonate. The reaction scheme is shown below.

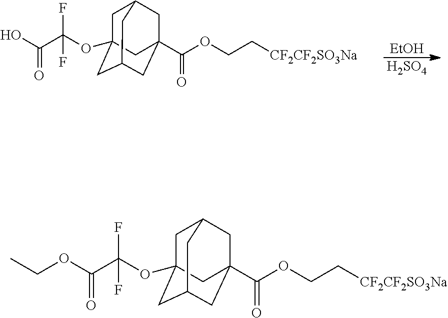

Into a reaction flask were charged 20.0 g of sodium 4-(3-(2-ethoxy-1,1-difluoro-2-oxoethoxy)adamantane-1-carbonyloxy)-1,1,2,2-tetrafluorobutane-1-sulfonate and 15.0 g of triphenylsulfonium bromide, 100 g of ion exchanged water and 100 g of dichloromethane, and the mixture was stirred at room temperature for 1 hour. After an organic layer was separated, the organic layer was washed with 100 g of ion exchanged water five times. Thereafter, a solvent was removed in vacuo to give 26.4 g of white solid of triphenylsulfonium 4-(3-(2-ethoxy-1,1-difluoro-2-oxoethoxy)adamantane-1-carbonyloxy)-1,1,2,2-tetrafluorobutane-1-sulfonate. The reaction scheme is shown below.

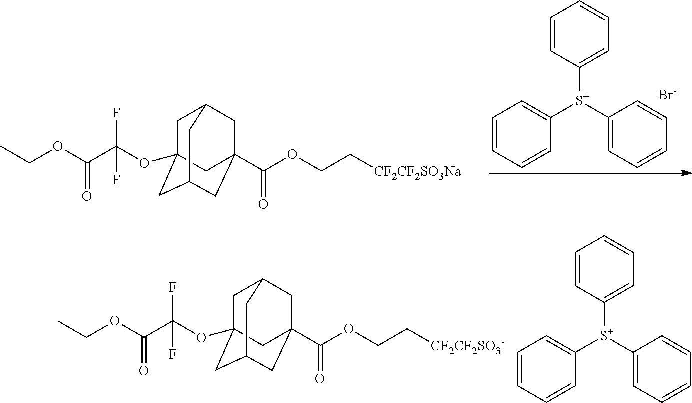

Note that an analysis was carried out with regard to triphenylsulfonium 4-(3-(2-ethoxy-1,1-difluoro-2-oxoethoxy)adamantane-1-carbonyloxy)-1,1,2,2-tetrafluorobutane-1-sulfonate using ¹H-NMR. As a result, the obtained chemical shift was as follows: ¹H-NMR [σppm (DMSO): 1.24-1.29 (3H,m), 1.36-1.45 (4H,m), 1.53-1.62 (4H,m), 1.84-2.01 (4H,m), 4.08-4.13 (2H,m), 7.76-7.89 (15H,m)], ¹⁹F-NMR [σppm (DMSO): 58.82 (m)]. Accordingly, the analysis confirmed that the sample was a compound of interest. The purity was no less than 99 wt %.

### Synthesis Example 5

Triphenylsulfonium 4-(4-(2-tertiarybutoxy-2-oxoethoxy)cyclohexanecarbonyloxy)-1,1,2,2-tetrafluorobutane-1-sulfonate (hereinafter, may be also referred to as “(A-4)”) represented by the following formula was synthesized by an operation similar to Synthesis Examples 1 and 2 using 4-hydroxycyclohexanecarboxylic acid in place of 3-hydroxyadamantane-1-carboxylic acid as a starting material.

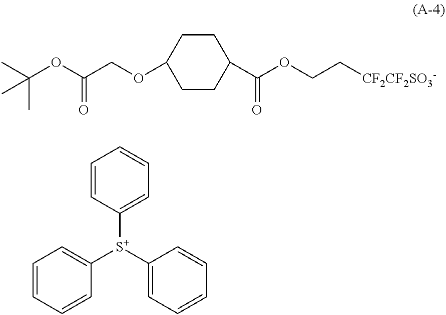

### Synthesis Example 6

Triphenylsulfonium 1,1,2,2-tetrafluoro-4-(4-(2,2,2-trifluoroacetoxy)cyclohexanecarbonyloxy)-butane-1-sulfonate (hereinafter, may be also referred to as “(A-5)”) represented by the following formula was synthesized in an operation similar to Synthesis Examples 1 and 3 using 4-hydroxycyclohexanecarboxylic acid in place of 3-hydroxyadamantane-1-carboxylic acid as the starting material.

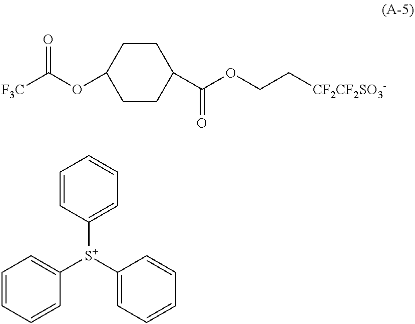

### Synthesis Example 7

Triphenylsulfonium 4-(4-(2-ethoxy-1,1-difluoro-2-oxoethoxy)cyclohexanecarbonyloxy)-1,1,2,2-tetrafluorobutane-1-sulfonate (hereinafter, may be also referred to as “(A-6)”) represented by the following formula was synthesized in an operation similar to Synthesis Examples 1 and 4 using 4-hydroxycyclohexanecarboxylic acid in place of 3-hydroxyadamantane-1-carboxylic acid as the starting material.

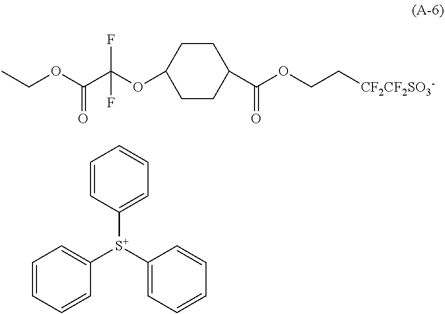

### <Synthesis of (B) Polymer>

### Synthesis Example 8

A monomer solution was prepared by dissolving 34.68 g (40 mol %) of the following compound (S-1), 45.81 g (40 mol %) of the following compound (S-3) and 6.71 g (10 mol %) of compound (S-4) into 200 g of 2-butanone, and further charging thereto 4.23 g of 2,2′-azobis(2-methylpropionitrile). A 1,000 mL three-neck flask into which 12.80 g (10 mol %) of the following compound (S-2) and 100 g of 2-butanone were charged was purged with nitrogen for 30 min. After the nitrogen-purge, a reaction tank was heated to 80° C. with stirring and the monomer solution prepared beforehand was added dropwise over 3 hrs using a dripping funnel. The time point at which the dropwise addition was started was defined as a polymerization starting time, and the polymerization reaction was performed for 6 hrs. After completing the polymerization, the polymerization solution was cooled to no higher than 30° C. by water cooling and the polymerization solution was charged into 4,000 g of methanol and deposited white powder was filtered off. The filtered white powder was dispersed in 400 g of methanol into a slurry form and washed. Thereafter, operation of refiltration was carried out twice. The obtained white powder was dried in vacuo at 50° C. for 17 hrs to give (B-1) a copolymer (90 g, yield: 90%). The Mw of the copolymer (B-1) was 6,136 and the Mw/Mn was 1.297. As a result of an analysis by ¹³C-NMR, the contents of the structural units derived from the compound (S-1), the compound (S-2), the compound (S-3) and the compound (S-4) were 40.4, 8.9, 41.0 and 9.7 (mol %), respectively.

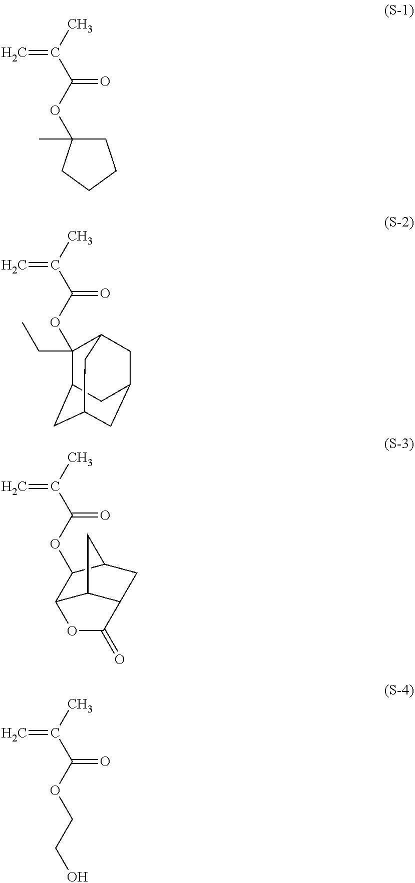

### <Synthesis of (C) Fluorine Atom-Containing Polymer>

### Synthesis Example 9

A monomer solution was prepared by dissolving 37.41 g (40 mol %) of the following compound (S-5) and 62.59 g (60 mol %) of compound (S-6) into 100 g of 2-butanone and further charging thereto 4.79 g of 2,2′-azobis (2-methylpropionitrile). A 1,000 mL three-neck flask into which 100 g of 2-butanone was charged was purged with nitrogen for 30 min. After the nitrogen-purge, a reaction tank was heated to 80° C. with stirring, the monomer solution prepared beforehand was added dropwise over 3 hrs using a dripping funnel. The time point at which the dropwise addition was started was defined as a polymerization starting time, and the polymerization reaction was performed for 6 hrs. After the completing of the polymerization, 150 g of 2-butanone was removed in vacuo from a polymerization solution. After cooling to no higher than 30° C., the polymerization solution was charged into a mixed solvent of 1900 g of methanol and 100 g of ultra pure water to filter off deposited white powder. 100 g of the filtered white powder was dispersed in methanol into a slurry form and was washed. Thereafter, operation of refiltration was carried out twice. The obtained white powder was dried in vacuo at 50° C. for 17 hrs to give (C-1) a copolymer (78 g, yield: 78%). The Mw of the copolymer (C-1) was 6,920 and the Mw/Mn was 1.592. As a result of analysis by ¹³C-NMR, the contents of the structural units derived from the compound (S-5) and the compound (S-6) were 40.8 and 59.2 (mol %), respectively.

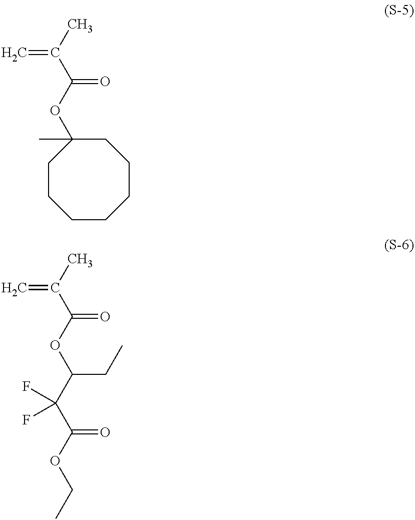

### <Preparation of Radiation-Sensitive Resin Composition>

Hereinafter, each component used for preparation in Examples and Comparative Examples will be shown in detail.

### [Other Acid Generating Agents]

A-7: triphenylsulfonium 2-bicyclo[2.2.1]hept-2-yl-1,1,2,2-tetrafluoroethanesulfonate
A-8: triphenylsulfonium 2-(adamantan-1-yl)-1,1-difluoroethane-1-sulfonate

A-9: triphenylsulfoniumperfluoro-n-butane-sulfonate

Other acid generating agents referred to in the above (A-7) to (A-9) are each represented by the following formulae.

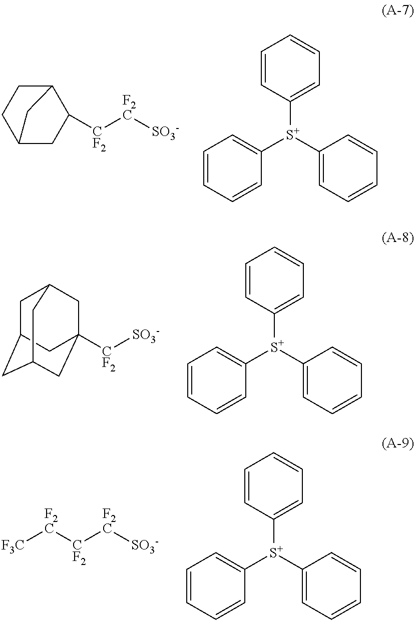

### <Diffusion Control Agent>

E-1: tert-amyl-4-hydroxy-1-piperidine carboxylate represented by the following formula

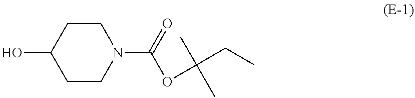

### <Solvent>

H-1: propyleneglycolmonomethyl ether acetate represented by the following formula
H-2: cyclohexanone represented by the following formula

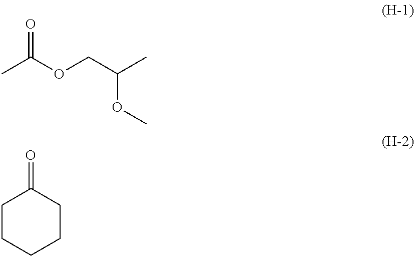

### Examples 1 to 18 and Comparative Examples 1 to 6

Using each component having the type and amounts blended shown in Tables 1-1 and 1-2, mixing with 1,750 parts by mass of (H-1) and 750 parts by mass of (H-2) as solvents gave each radiation-sensitive resin composition. It is noted that “-” in Tables 1-1 and 1-2 denotes that corresponding components were not used.

### <Evaluations>

Using each radiation-sensitive resin composition of Examples 1 to 18 and Comparative Examples 1 to 6, the following characteristics were evaluated. The evaluation results are shown together in Tables 1-1 and 1-2.

### [LWR]

Using a silicon wafer provided with an ARC66 (manufactured by Nissan Chemical Industries, Ltd.) film having a film thickness of 1,050 Å formed on the surface thereof, each radiation-sensitive resin composition was coated on the substrate by spin coating. Exposure was carried out on a photoresist film having a film thickness of 0.10 μm formed by carrying out PB at 110° C. for 60 sec on a hot plate using a liquid immersion ArF excimer laser lithography device manufactured by Nikon Corporation (numerical aperture: 1.30), through a mask having a line-and-space pattern (1L/1S) with a target size of a line width of 48 nm. Thereafter, PEB was carried out at temperatures shown in Tables for 60 sec, and then development was carried out at 23° C. for 4 sec with a 2.38% by mass aqueous tetramethylammoniumhydroxide solution, followed by washing with water and drying to form a positive type resist pattern. In this process, an exposure dose at which a line-and-space pattern of 1:1 (1L/1S) with a line width of 48 nm was formed was designated as “optimal exposure dose”, and this optimal exposure dose was regarded as sensitivity. In the observation of the 1L/1S pattern with a line width of 48 nm resolved at the optimal exposure dose, line widths at arbitrary ten points were measured when observed from above the pattern using a SEM for critical dimension measurement: CG4000 manufactured by Hitachi, Ltd., and the variance of measurements expressed as a value in terms of the 3 Sigma was defined as “LWR”. Linearity of the pattern after development was determined to be more favorable as the LWR value is smaller.

### [MEEF]

The optimal exposure dose was determined by an operation similar to the evaluation item of the LWR described above except that an exposure dose was employed which provided a line having a line width of 50 nm formed using a mask having a line-and-space pattern (1L/1S) with a target size of a line width of 50 nm as a mask. Line widths of patterns resolved by a mask having a line-and-space pattern with a pitch of 100 nm in and with the target sizes of line width at the optimal exposure dose being 46 nm, 48 nm, 50 nm, 52 nm and 54 nm were determined. The results were shown by plotting the target sizes along the abscissa with respect to the line widths on the ordinate, and the slope obtained by a least-aqueous method was defined as “MEEF”. When the slope was more approximate to 1, mask reproducibility was determined to be more favorable.

### [Development Defects]

A resist pattern was formed by an operation similar to the evaluation of LWR described above except that exposure was carried out such that an interval between a shot and a neighboring shot was 1 mm on the entirety of the wafer using a mask having a 1L/1S pattern with a target size of 48 nm on the entire surface. Sites not exposed with light between shots were inspected by a defect inspection system KLA2810 and the number of defects was evaluated per inspection area of 1 cm². When the number of defects was less, the development defects were determined to be more inhibited.

TABLE 1-1

| | | | | | (C) Fluorine | | | | | | | |
|---|---|---|---|---|---|---|---|---|---|---|---|---|
| | | | | | atom-containing | | Acid diffusion | | | | | |
| | (A) Component | | (B) Polymer | | polymer | | control agent | | | | | |
| | | Amount | | Amount | | Amount | | Amount | | | | |
| | | blended | | blended | | blended | | blended | | | | Develop- |
| | | (parts | | (parts | | (parts | | (parts | PEB | | | ment |
| | Type | by mass) | Type | by mass) | Type | by mass) | Type | by mass) | (° C.) | LWR | MEEF | defects |
| Example 1 | A-1 | 10 | B-1 | 100 | — | — | E-1 | 15 | 105 | 4.5 | 2.5 | 6 |
| Example 2 | A-2 | 10 | B-1 | 100 | — | — | E-1 | 15 | 105 | 4.4 | 2.6 | 5 |
| Example 3 | A-3 | 10 | B-1 | 100 | — | — | E-1 | 15 | 105 | 4.5 | 2.5 | 6 |
| Example 4 | A-4 | 10 | B-1 | 100 | — | — | E-1 | 15 | 100 | 5 | 3.8 | 4 |
| Example 5 | A-5 | 10 | B-1 | 100 | — | — | E-1 | 15 | 100 | 4.9 | 3.6 | 3 |
| Example 6 | A-6 | 10 | B-1 | 100 | — | — | E-1 | 15 | 100 | 4.9 | 3.7 | 5 |
| Example 7 | A-1 | 10 | B-1 | 100 | C-1 | 3 | E-1 | 15 | 105 | 4.4 | 2.7 | 6 |
| Example 8 | A-2 | 10 | B-1 | 100 | C-1 | 3 | E-1 | 15 | 105 | 4.4 | 2.6 | 8 |
| Example 9 | A-3 | 10 | B-1 | 100 | C-1 | 3 | E-1 | 15 | 105 | 4.5 | 2.7 | 8 |
| Example 10 | A-4 | 10 | B-1 | 100 | C-1 | 3 | E-1 | 15 | 100 | 4.9 | 3.5 | 5 |
| Example 11 | A-5 | 10 | B-1 | 100 | C-1 | 3 | E-1 | 15 | 100 | 4.9 | 3.4 | 6 |
| Example 12 | A-6 | 10 | B-1 | 100 | C-1 | 3 | E-1 | 15 | 100 | 4.9 | 3.6 | 7 |
| Example 13 | A-1/A-9 | 8/2 | B-1 | 100 | C-1 | 3 | E-1 | 15 | 105 | 4.5 | 2.8 | 4 |
| Example 14 | A-2/A-9 | 8/2 | B-1 | 100 | C-1 | 3 | E-1 | 15 | 105 | 4.5 | 2.8 | 5 |
| Example 15 | A-3/A-9 | 8/2 | B-1 | 100 | C-1 | 3 | E-1 | 15 | 105 | 4.6 | 2.8 | 6 |
| Example 16 | A-4/A-9 | 8/2 | B-1 | 100 | C-1 | 3 | E-1 | 15 | 100 | 4.9 | 3.4 | 6 |
| Example 17 | A-5/A-9 | 8/2 | B-1 | 100 | C-1 | 3 | E-1 | 15 | 100 | 4.8 | 3.7 | 5 |
| Example 18 | A-6/A-9 | 8/2 | B-1 | 100 | C-1 | 3 | E-1 | 15 | 100 | 4.8 | 3.6 | 3 |

TABLE 1-2

| | | | | | (C) Fluorine | | | | | | | |
|---|---|---|---|---|---|---|---|---|---|---|---|---|
| | | | | | atom-containing | | Acid diffusion | | | | | |
| | (A) Component | | (B) Polymer | | polymer | | control agent | | | | | |
| | | Amount | | Amount | | Amount | | Amount | | | | |
| | | blended | | blended | | blended | | blended | | | | Develop- |
| | | (parts | | (parts | | (parts | | (parts | PEB | | | ment |
| | Type | by mass) | Type | by mass) | Type | by mass) | Type | by mass) | (° C.) | LWR | MEEF | defects |
| Comparative Example 1 | A-7 | 10 | B-1 | 100 | — | — | E-1 | 15 | 100 | 5 | 3.8 | 20 |
| Comparative Example 2 | A-8 | 10 | B-1 | 100 | — | — | E-1 | 15 | 105 | 4.2 | 2.9 | 51 |
| Comparative Example 3 | A-9 | 10 | B-1 | 100 | — | — | E-1 | 15 | 100 | 6 | 4.9 | 7 |
| Comparative Example 4 | A-7 | 10 | B-1 | 100 | C-1 | 3 | E-1 | 15 | 100 | 5.1 | 3.8 | 35 |
| Comparative Example 5 | A-8 | 10 | B-1 | 100 | C-1 | 3 | E-1 | 15 | 105 | 4.2 | 2.8 | 112 |
| Comparative Example 6 | A-9 | 10 | B-1 | 100 | C-1 | 3 | E-1 | 15 | 100 | 6.3 | 5.1 | 9 |

As is clear from the results shown in Tables 1-1 and 1-2, the radiation-sensitive resin compositions of Examples 1 to 18 that include the acid generating agent (A) were not only more favorable in MEEF and LWR but also accompanied by reduced development defects as compared with the radiation-sensitive resin compositions of Comparative Examples 1 to 6 that include no acid generating agent (A). It is to be noted Examples 1 to 3, 7 to 9 and 13 to 15 in which an organic acid was used having a polycyclic hydrocarbon group introduced into their anion moieties were more favorable in MEEF and LWR than Examples 4 to 6, 10 to 12 and 16 to 18 in which an organic acid was used having a monocyclic hydrocarbon group introduced thereto. It is considered that it results from an appropriate control of a diffusion length of an organic acid since a polycyclic hydrocarbon group is more bulky than a monocyclic hydrocarbon group. Accordingly, it can be inferred that introduction of a bulky polycyclic hydrocarbon group is preferable in order to adjust a diffusion length.

In addition, in Comparative Examples 1, 2, 4 and 5 in which an organic acid having no bonds cleavable by an acid or a base was used, although a polycyclic hydrocarbon group was introduced, development defects occurred at a high level although MEEF and LWR were comparative or only a little inferior to those in Examples. It is considered that these features result from low miscibility with an alkaline developer and aggregation occurred in the developing step since a bond cleavable by an acid or a base was not introduced whereas a diffusion length was appropriately controlled and lithography property was favorable by introduction of a polycyclic hydrocarbon group into the organic acid. Furthermore, in Comparative Examples 3 and 6 in which neither a cyclic hydrocarbon group nor an organic group including a cleavable bond was introduced, the results exhibited inferior MEEF and LWR whereas few development defects were found. It is considered that these features result from an increased diffusion length, thereby leading to deterioration of the lithography property since the organic acid had no bulky structure whereas the organic acid was easily removed in the developing step due to having a comparatively small hydrophobic moiety.

According to the embodiments of the present invention, a radiation-sensitive resin composition can be provided which reduces generation of development defects also in a liquid immersion lithography process and can form a resist pattern excellent in MEEF and LWR. Therefore, the composition is useful as a chemical amplification type resist.

Obviously, numerous modifications and variations of the present invention are possible in light of the above teachings. It is therefore to be understood that within the scope of the appended claims, the invention may be practiced otherwise than as specifically described herein.

1. A radiation-sensitive resin composition comprising:
an acid generating agent to generate an organic acid by irradiation with a radioactive ray, the organic acid having a cyclic hydrocarbon group and an organic group including a bond that is cleavable by an acid or a base to produce a polar group.

2. The radiation-sensitive resin composition according to claim 1, wherein the organic acid is represented by a following formula (I):
Z—R¹—X—R²—(R³)ₙ (I)
wherein,
Z represents an organic acid group;
R¹ represents an alkanediyl group, wherein a part or all of hydrogen atom in the alkanediyl group represented by R¹ are optionally substituted by a fluorine atom;
X represents a single bond, O, OCO, COO, CO, SO₃ or SO₂;
R² represents a cyclic hydrocarbon group;
R³ represents a monovalent organic group having a functional group represented by a following formula (x); and
n is an integer of 1 to 3, wherein in a case where R³ is present in a plurality of number, R³s present in the plurality of number are a same or different,
—R³¹-G-R¹³ (x)
wherein, in the formula (x),
R³¹ represents a single bond or a bivalent linking group;
G represents an oxygen atom, an imino group, —NR¹³¹—, —CO—O—*, —O—CO—* or —SO₂—O—*, wherein the oxygen atom represented by G excludes an oxygen atom directly bonded to a carbonyl group or a sulfone group;
each of R¹³¹ and R¹³ represents an acid-dissociable group or
a base-dissociable group; and
“*” denotes a site bound to R¹³.

3. The radiation-sensitive resin composition according to claim 2, wherein Z is SO₃H.

4. The radiation-sensitive resin composition according to claim 2, wherein R¹ is represented by a following formula (1): 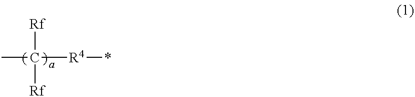
wherein,
each of Rf independently represents a hydrogen atom, a fluorine atom, or an alkyl group in which a part or all of hydrogen atoms are substituted by a fluorine atom;
R⁴ represents an alkanediyl group;
“a” is an integer of 1 to 8, wherein in a case where “a” is 2 or more, Rfs present in a plurality of number are a same or different, and a case where all the Rfs are a hydrogen atom is excluded; and
“*” denotes a site bound to X.

5. The radiation-sensitive resin composition according to claim 2, wherein R³ is a structure represented by a following formula (2) : 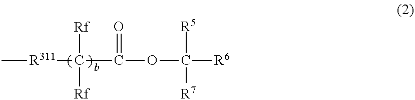
wherein,
R³¹¹ represents a single bond or a bivalent linking group;
Rf is as defined in the above formula (1);
R⁵ to R⁷ each independently represent an alkyl group having 1 to 4 carbon atoms or an alicyclic hydrocarbon group having 4 to 20 carbon atoms;
R⁶ and R⁷ are optionally taken together represent a bivalent alicyclic hydrocarbon group having 4 to 20 carbon atoms together with the carbon atom to which R⁶ and R⁷ bond;
b is an integer of 0 to 8, wherein in a case where b is 2 or more, Rfs present in a plurality of number are a same or different, and a case where all the Rfs are a hydrogen atom is excluded.

6. The radiation-sensitive resin composition according to claim 2, wherein R³ comprises a structure represented by a following formula (3) or (4): 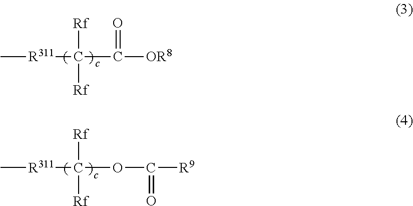
wherein, in the formulae (3) and (4),
R³¹¹ is as defined in the above formula (2);
Rf is as defined in the above formula (1);
R⁸ represents an alkyl group having 1 to 10 carbon atoms in which a part or all of hydrogen atoms are substituted by a fluorine atom or a group represented by a following formula (5), (6) or (7);
R⁹ represents an alkyl group having 1 to 10 carbon atoms in which a part or all of hydrogen atoms are substituted by a fluorine atom;
c is an integer of 0 to 4, wherein in a case where c is 2 or more, Rfs present in a plurality of number are a same or different, and a case where all Rfs are a hydrogen atom is excluded: 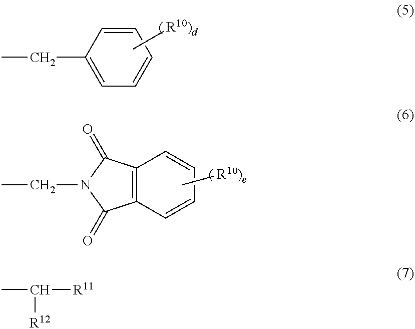
wherein,
in the formulae (5) and (6),
R¹⁰ each independently represent a halogen atom, an alkyl group, an alkoxy group, an acyl group or an acyloxy group having 1 to 10 carbon atoms;
d is an integer of 0 to 5;
e is an integer of 0 to 4, wherein in a case where R¹⁰ is present in a plurality of number, R¹⁰s present in the plurality of number are a same or different:
in the formula (7), R¹¹ and R¹² each independently represent a hydrogen atom or an alkyl group having 1 to 10 carbon atoms, wherein R¹¹ and R¹² are optionally taken together represent an alicyclic structure having 4 to 20 carbon atoms together with the carbon atom to which R¹¹ and R¹² bond.

7. The radiation-sensitive resin composition according to claim 2, wherein R² is represented by a following formula (8), (9) or (10): 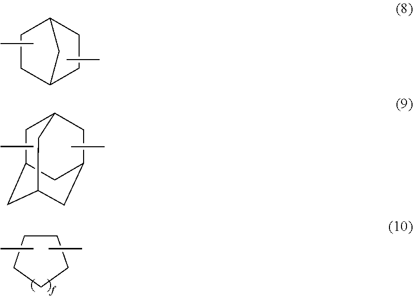
wherein, in the formula (10), f is an integer of 1 to 10.

8. The radiation-sensitive resin composition according to claim 2, wherein R² represents a polycyclic hydrocarbon group.

9. The radiation-sensitive resin composition according to claim 2, wherein the acid generating agent is a sulfonium salt compound or an iodonium salt compound of the organic acid represented by the above formula (I).

10. A method for forming a resist pattern, comprising:
providing the radiation-sensitive resin composition according to claim 1 on a substrate to form a photoresist film;
exposing the formed photoresist film through an immersion liquid; and
developing the exposed photoresist film to form a resist pattern.

11. An organic acid represented by a following formula (I) or a salt thereof:
Z—R¹—X—R²—(R³)ₙ (I)
wherein,
Z represents an organic acid group;
R¹ represents an alkanediyl group, wherein a part or all of hydrogen atoms of the alkanediyl group represented by R¹ are optionally substituted by a fluorine atom;
X represents a single bond, O, OCO, COO, CO, SO₃ or SO₂;
R² represents a cyclic hydrocarbon group;
R³ represents a monovalent organic group having a functional group represented by a following formula (x); and
n is an integer of 1 to 3, wherein in a case where R³ is present in a plurality of number, R³s present in the plurality of number are a same or different:
—R³¹-G-R¹³ (x)
wherein, R³¹ represents a single bond or a bivalent linking group;
G represents an oxygen atom, an imino group, —NR¹³¹—, —CO—O—*, —O—CO—* or —SO₂—O—*, wherein the oxygen atom represented by G excludes an oxygen atom directly bonded to a carbonyl group or a sulfone group;
each of R¹³¹ and R¹³ represents an acid-dissociable group or a base-dissociable group; and
“*” denotes a site bound to R¹³.

12. An acid generating agent that generates an organic acid represented by a following formula (I) by irradiation with a radioactive ray:
Z—R¹—X—R²—(R³)ₙ (I)
wherein,
Z represents an organic acid group;
R¹ represents an alkanediyl group, wherein a part or all of hydrogen atoms of the alkanediyl group represented by R¹ are optionally substituted by a fluorine atom;
X represents a single bond, O, OCO, COO, CO, SO₃ or SO₂;
R² represents a cyclic hydrocarbon group;
R³ represents an organic group having a monovalent functional group represented by a following formula (x); and
n is an integer of 1 to 3, wherein in a case where R³ is present in a plurality of number, R³s present in the plurality of number are a same or different:
—R³¹-G-R¹³ (x)
wherein, in the formula (x),
R³¹ represents a single bond or a bivalent linking group;
G represents an oxygen atom, an imino group, —NR¹³¹—, —CO—O—*, —O—CO—* or —SO₂—O—*, wherein the oxygen atom represented by G excludes an oxygen atom directly bonded to a carbonyl group or a sulfone group;
each of R¹³¹ and R¹³ represents an acid-dissociable group or a base-dissociable group; and
“*” denotes a site bound to R¹³.
